(19) United States
(12) Patent Application Publication
(10) Pub. No.: US 2015/0118747 A1
(43) Pub. Date: Apr. 30, 2015

(54) ELECTROSTRETCHED POLYMER MICROFIBERS FOR MICROVASCULATURE DEVELOPMENT

(71) Applicant: The Johns Hopkins University, Baltimore, MD (US)

(72) Inventors: Sharon Gerecht, Baltimore, MD (US); Shuming Zhang, Baltimore, MD (US); Sebastian F. Barreto Ortiz, Baltimore, MD (US); Hai-Quan Mao, Baltimore, MD (US)

(21) Appl. No.: 14/530,362

(22) Filed: Oct. 31, 2014

Related U.S. Application Data
(60) Provisional application No. 61/897,955, filed on Oct. 31, 2013.

(51) Int. Cl. C12N 5/00

(52) U.S. Cl. CPC C12N 5/0068; C12N 2535/00; C12N 2533/74; C12N 2533/50; C12N 2533/80
USPC 435/354; 435/398

(57) ABSTRACT

An in vitro model system that guides the development of microvasculature, recapitulating the detailed organization of both its cellular and a-cellular components is established. Use of electrostretched fibrin microfibers enables both endothelial layer organization and co-culture of supporting perivascular (mural) cells such as vascular smooth muscle cells and pericytes. The fiber curvature affects the circumferential deposition of endothelial-produced ECM independently of cellular organization and induces deposition of higher quantities of vascular ECM proteins. Further, a luminal multicellular microvascular structure is disclosed.

### CROSS REFERENCE TO RELATED APPLICATION

This claims benefit of U.S. Provisional Application Ser. No. 61/897,955, filed Oct. 31, 2013, the entire contents of which is incorporated by reference herein.

This invention was made with U.S. Government support under government grants R01HL107938 and U54CA143868 awarded by the National Institutes of Health, and grant number DMR-0748340 awarded by the National Science Foundation. The government has certain rights in this invention.

### FIELD OF INVENTION

This technology is a microvascular structure developed in vitro using a novel polymer microfiber with tunable diameter and internal and external longitudinal alignment.

### BACKGROUND

The development of artificial microvascular vessels is of critical importance in the fields of cardiovascular diseases, cancer growth, and tissue engineering of vascularized organs. In tissue engineering there is currently a limit on the size of tissues that can be constructed in vitro due to the diffusion limit of nutrients into developing organs. As such, only thin tissues like skin grafts or avascular tissues like cartilage can be successfully engineered in vitro, with larger constructs requiring the existence of a vascular network within itself to supply the necessary nutrients for cell survival and to avoid necrotic areas post-implantation (Huang, G. Y., et al., Biofabrication, 2011. 3(1):012001, and Auger, F. A., et al., Annual Rev Biomed Engineering, 2013. 15(1):177-200. Development of functional microvascular vessels requires complex interactions between endothelial cells (ECs), perivascular cells, and the extracellular matrix (ECM) (Jain R K, Nat Med, 2003; 9: 685-693; Carmeliet P and Jain R K, Nature, 2000; 407: 249-257). The ECM is comprised of an abundance of nanometer sized macromolecular ECM proteins. As such, many of the physical interactions between vascular cells and macromolecular components of the ECM occur at the sub-micron scale. EC adhesion to the ECM initiates the angiogenic cascade (Hynes R O, J. Thromb Haemost, 2007; 5: 32-40). Cells detect and respond to the nanoarchitecture of their microenvironment by cytoskeletal reorganization and activated signaling cascades to regulate fundamental cell behaviors. Indeed, it has been shown that surfaces with nano-scale line-grating features affect EC adhesion, alignment, and elongation (Ranjan A, and Webster T, Nanotechnology, 2009; 20: 305102; Liliensiek S, et al., Biomaterials, 2010; 31: 5418-5426; Bettinger C J, et al., Adv Mater, 2008; 20: 99-103; Lu J, et al., Acta Biomater, 2008; 4: 192-201).

Arterioles are the blood vessels found immediately before capillaries, ranging from tens to hundreds of microns in diameter. ECs, sitting on their basal lamina, comprise the innermost lining of the vessels, this layer containing mainly collagen type IV (Col IV), fibronectin (Fn), laminin (Lmn), and heparin sulfate proteoglycan (Roy, S., et al., Current Eye Research, 2010; 35(12):1045-1056). This is followed by a layer of subendothelial connective tissue and an internal elastic lamina (Hibbs, R. G., et al., Am Heart J, 1958; 56(5):662-670; Yen, A. and I. M. Braverman, J Investig Dermatol, 1976; 66(3):131-142; Weber, K. and O. Braun-Falco, Archiv für dermatologische Forschung, 1973; 248(1):29-44), which are composed of different ECM proteins, mainly of collagens such as type I and III (Col I, Col III), and elastin (Eln) (Yen, A. J Investig Dermatol, 1976; Tsamis, A., et al., J Royal Society Interface, 2013; 10(83):20121004. These layers together make the tunica intima of blood vessels. Mural cells, along with their ECM, make up the tunica media (or middle layer), a layer of perivascular cells increasing in thickness with vessel size (Yen, A. J Investig Dermatol, 1976; Marieb, E. N. and K. Hoehn, Human anatomy & physiology. 2007: Pearson Education; Standring, S., Gray's anatomy. The anatomical basis of clinical practice, 2008. 39), and provide the contractility necessary for vasoreactivity. In the smallest of arterioles the perivascular cells are pericytes, which increase in confluency with vessels size and eventually are replaced by vascular smooth muscle cells (vSMCs) (Yen, A. J Investig Dermatol, 1976; Weber, K., 1973; Marieb, E. N. and K. Hoehn, 2007: Pearson Education; Standring, S., Gray's anatomy, 2008:39). The tunica media in arterioles has a primarily circumferential orientation of the vSMCs, which is necessary for vasoconstriction. Opposite to arterioles, venules collect blood from the capillary beds and are also supported by perivascular cells, though here the tunica media does not follow a circumferential organization and the ECM layers are less robust than in arterioles ((Hibbs, R. G., et al., Am Heart J, 1958; Yen, A. and I. M. Braverman, J Investig Dermatol, 1976; Marieb, E. N. and K. Hoehn, 2007: Pearson Education; Standring, S., Gray's anatomy, 2008:39; Jain, R. K., Nature medicine, 2003; 9(6): p. 685-693). On top of the tunica media layer lays the tunica adventitia, though it is only present in larger blood vessels (Carmeliet P and Jain R K, Nature, 2000; 407: 249-257; Bruce Alberts A J, et al., Molecular Biology of the Cell, 2002, New York: Garland Science; Jain R K, Nature Medicine, 2003; 9: 685-693; Schwartz S M, and Benditt E P, American J Pathology, 1972; 66: 241; Schriefl A J, et al., Journal of The Royal Society Interface, 2012; 9: 1275-1286; Tsamis A, et al., Journal of The Royal Society Interface; 2013; 10; Canham, P B, et al., Cardiovascular 1989; 23: 973-982; Finlay H, et al., Journal of vascular research; 1995; 32: 301-312; Movat H Z, et al., Experimental and Molecular Pathology, 1963; 2: 549-563). The specific composition as well as the organization and arrangement of both cellular and ECM components in each layer are necessary for proper microvasculature development, maturation, stability, and function (Jain R K, Nat Med, 2003; 9: 685-693; Carmeliet P and Jain R K, Nature, 2000; 407: 249-257).

It has been shown that in microvasculature EC nuclei and cytoskeleton are aligned in the direction of blood flow. However, studies on microvasculature have not analyzed the organization of different ECM components in detail (Movat H Z, and Fernando N V P, Experimental and Molecular Pathology, 1963; 2: 549-563; Hibbs R G, et al., American Heart Journal, 1958; 56: 662-670; Fernando N V, and Movat H Z, Experimental and Molecular Pathology, 1964; 3: 1-9). The literature that has studied the particular organization of the ECM in native blood vessels is focused on analyzing the elastin and collagen structures in the aorta and other large arteries (Schwartz S M, and Benditt E P, American J Pathology, 1972; 66: 241; Schriefl A J, et al., Journal of The Royal Society Interface, 2012; 9: 1275-1286; Tsamis A, et al., Journal of The Royal Society Interface; 2013; 10; Canham, P B, et al., Cardiovascular, 1989; 23: 973-982; Finlay H, et al., Journal of vascular research, 1995; 32: 301-312; Halloran B G, et al., Journal of Surgical Research, 1995; 59: 17-22). These studies have shown that the structural organization of collagen varies not only between the three layers of the vasculature, but also varies with vessel size and specific location in the body (Tsamis A, et al., Journal of The Royal Society Interface, 2013; 10).

Overall, the studies agree that each tunica possesses at least two different families of collagen fibrils, with distinctly different organizations. The general arrangement has been found to be close to axial in the adventitia, outer layers having a more pronounced axial orientation transitioning to a circumferential alignment in inner layers (Finlay H, et al., Journal of vascular research, 1995; 32: 301-312). In contrast, the medial layer has been shown to have a nearly perfect circumferential order (Schriefl A J, et al., Journal of The Royal Society Interface, 2012; 9: 1275-1286; Tsamis A, et al., Journal of The Royal Society Interface; 2013; 10; Canham, P B, et al., Cardiovascular, 1989; 23: 973-982; Finlay H, et al., Journal of vascular research, 1995; 32: 301-312). Studies also agree both the internal and external elastic lamina are fenestrated, though both axial and circumferential organization of these layers has been reported (Movat H Z, and Fernando N V P, Experimental and Molecular Pathology, 1963; 2: 549-563; Moore D H, and Ruska H, J Biophysical and Biochemical Cytology, 1957; 3: 457-462). On the other hand, the subendothelium has been found to have a more varied composition. It has been described as a multilayered fabric of collagen, containing distinct layers of both longitudinally and circumferentially aligned fibers (Schwartz S M, and Benditt E P, American J Pathology, 1972; 66: 241; Canham, P B, et al., Cardiovascular, 1989; 23: 973-982; Finlay H, et al., Journal of vascular research, 1995; 32: 301-312; Gasser T C, et al., J Royal Society Interface, 2006; 3: 15-35); some studies suggesting a layer of longitudinally aligned ECM directly under the media layer, followed by a helically arranged region of connective tissue beneath, and a thin circumferentially aligned layer next to the lumen (Finlay H, et al., Journal of vascular research, 1995; 32: 301-312). However, these findings were all made on large arteries with diameters in the millimeter range.

The creation of clinically relevant functional microvascular conduits (i.e. arterioles and venules) remains a challenge. To date, only a few studies have attempted to model or recreate microvasculature in a full 3D setting in vitro (Miller J S, et al., Nat Mater, 2012; 11: 768-774; Zheng Y, et al., PNAS USA, 2012; 109: 9342-9347; Neumann T, et al., Microvascular Research, 2003; 66: 59-67; Norotte, C., et al., Biomaterials, 2009; 30 (30): p. 5910-5917), though none has achieved a fully functional microvascular structure recapitulating both the cellular and ECM protein organization in the multilayer formation present in native vasculature. The majority of models have either focused on the development of capillary beds in natural or synthetic hydrogels (Yee D, et al., Tissue Engineering Part A, 2011; 1351-1361; Hanjaya-Putra D, et al., J Cellular Molecular Medicine, 2010; 14: 2436-2447; Meyer G T, et al., Anat. Rec., 1997; 249: 327-340; Bayless K J, Davis G E, J Cell Science, 2002; 115: 1123-1136; Bayless K J, American J Pathology, 2000; 156: 1673-1683; Grant D S, et al., Cell Press, 1989; 933-943; Sacharidou A, et al., Cells Tissues Organs, 2011; 195: 122-143; Davis G E, and Camarillo C W, Experimental Cell Research 1996; 224: 39-51; Kim D J, et al., Blood, 2013; 121(17):3521-30; Hanjaya-Putra D, et al., Blood, 2011; 118: 804-815; Lutolf M, and Hubbell J, Nature biotechnology, 2005; 23: 47-55; Moon J J, et al., Biomaterials, 2010; 31: 3840-3847), decellularized matrices (Hielscher A C, et al., American Journal of Physiology—Cell Physiology, 2012; 302: C1243-C1256; Soucy P A, and Romer L H, Matrix Biology, 2009; 28: 273-283), and electrospun polymer scaffolds (Pham Q P, et al., Tissue Eng, 2006; 12: 1197-1211; Kumbar S G, et al., Biomed Mater, 2008; 3: 034002), or have instead aimed to create vascular grafts typically >3 mm in diameter (Aper T, Schmidt et al., European Journal of Vascular and Endovascular Surgery, 2007; 33: 33-39; Gui L, Tissue engineering Part A, 2009; 15: 2665-2676; Swartz D D, et al., American Journal of Physiology-Heart and Circulatory Physiology, 2005; 288: H1451-H1460; Kaushal S, et al., Nature medicine, 2001; 7: 1035-1040; Cho S-W, et al., Annals of surgery, 2005; 241: 506; L'heureux N, et al., The FASEB Journal, 1998; 12: 47-56). While these models enabled study of the ECM-driven molecular mechanisms that regulate EC tubulogenesis, they mostly support spontaneous and random tubulogenesis (size, shape, organization, etc.). Recent work employs micro-patterning and demonstrates an organized vascular network structure within hydrogels, some of which are able to recruit vascular smooth muscle cells (vSMCs) (Baranski J D, et al., Proc Natl Acad Sci USA, 2013; Miller J S, et al., Nat Mater, 2012; 11: 768-774; Zheng Y, et al., Proc Natl Acad Sci USA, 2012; 109: 9342-9347). Nonetheless, these systems have limited control over topographical cues presented by the ECM and impart a barrier for the high-resolution, dynamic, and detailed study of vascular organization as well as specific cell-ECM and multi-cellular interactions.

Tubular polymeric scaffolds have the potential to provide a better and more sophisticated platform to study the microvasculature, but currently are obtainable with diameters in the millimeter range (Melchiorri A J, et al., Tissue Eng Part B Rev, 2012; Fioretta E S, et al., Macromol Biosci, 2012; 12: 577-590; Gui L, et al., Tissue Eng Part A, 2009; 15: 2665-2676) and are mostly used to study graft's mechanical strength (Wu W, et al., Nat Med, 2012; 18: 1148-1153; Huynh T, and Tranquillo R, Annals of Biomedical Engineering, 2010; 38: 2226-2236; Lee K-W, et al., PNAS, 2011; 108: 2705-2710). To recapitulate the microvasculature in vitro, which has not previously been obtainable, the tubular scaffolds must exhibit a physiologically-relevant diameter and sub-micron topography with sufficient mechanical properties, be biocompatible, mediate specific cell adhesion, allow tubular vessel formation, and, support multi-cellular interactions, thus generating microvessels that mimic natural structure and properties.

Endothelial colony forming cells (ECFCs), a subpopulation of endothelial progenitor cells, are known for their proliferative capacity and contribution to functional vessels (Critser P J, and Yoder M C, Curr Opin Organ Transplant, 2010; 15: 68-72; Yoder M C, J of Thrombosis and Haemostasis, 2009; 7: 49-52; Yoder M C, et al., Blood, 2007; 109: 1801-1809). Recently, we revealed that ECFCs deposit ECM proteins, namely collagen IV, fibronectin and laminin, and also assemble ECM into web-like structures when cultured on Petri dishes (Kusuma S, et al., FASEB J, 2012; 26: 4925-4936). This finding suggests an important role for ECM production by ECFCs in the process of vascular assembly that has not yet been identified.

The cellular and ECM composition, as well as their specific structural organization, varies greatly in blood vessels of different type, size, and function. While postcapillary venules (10-30 μm) are formed by ECs and their basal lamina, along with scattered pericytes, and are semipermeable like capillaries (Standring S, 2008, Gray's anatomy), larger venules (larger than 50 μm in diameter) have a muscle layer and a thin adventitia (Standring S, 2008). Both arterioles of 100 μm to 300 μm in diameter and muscular arteries (300 μm to 1 cm) have an aligned endothelium sitting on its basal lamina, surrounded by a layer of connective tissue. This layer is followed by a well-defined fenestrated internal elastic membrane (with ECM occupying void spaces in the fenestrae) and a developed tunica media composed of several layers of circumferentially oriented vSMCs and ECM (mainly collagenous and elastic fibrils). These arteries are the main vessels in charge of restricting blood flow to capillary beds via vSMC constriction in response to neural or chemical stimuli (Standring S, 2008; Marieb E, Hoehn K, Human Anatomy & Physiology (8 ed., 2010), Boston, Mass.: Pearson Benjamin Cummings). Even though the ECM is known to be circumferentially oriented in the tunica media, the specific alignment of each layer in the tunica intima remains unclear; several studies report a mix of circumferential orientation close to the lumen and axial orientation close to the media (Schwartz S M, and Benditt E P, American J Patholo, 1972; 66: 241-264; Schriefl A J, et al., Journal of The Royal Society Interface, 2012; 9: 1275-1286; Tsamis A, et al., Journal of The Royal Society Interface; 2013; Movat H Z, et al., Experimental and Molecular Pathology, 1963; 2: 549-563; Fernando N V, and Movat H Z, Experimental and Molecular Pathology, 1964; 3: 1-9). However, it is known that circumferentially aligned ECM is necessary for vessels to be able to withstand the circumferential stress resulting from the distending pressure of blood flow. These facts highlight the importance of having both circumferential alignment of ECM and several layers of vSMCs, an aspect not found in current microvasculature models. Furthermore, the organization of other ECM components, such as fibronectin and laminin, remains widely uninvestigated. There is a need for a model to study the microvasculature that recapitulates endothelial cell and perivascular cell alignment and ECM deposition for generating microvessels.

### SUMMARY OF THE INVENTION

The present invention is a three-dimensional (3D) fibrin microfiber scaffold for a novel in vitro model of the microvasculature that recapitulates endothelial cell alignment and ECM deposition. The microfiber scaffold allows the sequential co-culture of endothelial cells and other cells, such as perivascular cells. This model establishes that the fiber curvature affects the circumferential deposition of ECM from endothelial cells independently of cellular organization. The invention further presents a multicellular microvascular structure with an organized endothelium and multicellular perivascular tunica media. The present invention provides unique opportunities to assess microvasculature development and regeneration.

As embodied and fully described, the invention relates to a tubular polymeric scaffold, for example a polymer hydrogel microfiber, comprised of electrostretched polymer nanofibers. The polymer preferably is alginate, fibrin (fibrinogen), gelatin, hyaluronic acid, or combinations thereof. More preferably, the polymer is fibrin. An electrostretched polymer microfiber serves as a template for the step-wise creation of microvasculature in vitro.

An embodiment of the invention relates to an electrostretched polymer microfiber that has uniform and tunable diameter, while preserving an aligned internal and external nanotopography. The polymer preferably is alginate, fibrin (fibrinogen), gelatin, hyaluronic acid, or combinations thereof. In a particular embodiment, the polymer is fibrin. Preferably, the polymer microfiber has a diameter of about 500 μm or less, more preferably from about 100 μm to about 450 μm.

In an embodiment of the invention, the electrostretched polymer microfiber generates a micro-cylindrical fiber with a line grating nanotopography. The microfiber enables both endothelial layer organization and co-culture with a second cell type, for example supporting mural cells (perivascular cells).

In some embodiments, the invention relates to a polymer microfiber seeded with endothelial progenitor cells, such as an endothelial colony forming cells. The endothelial cells adhere to the surface of the fibrin microfiber, and align longitudinally with the polymer microfiber. The attached endothelial cells deposit extracellular matrix (ECM) circumferentially organized depending on the size of the microfiber. The extracellular matrix is composed of proteins that encircle (wrap around) the microfiber along the fiber circumference, perpendicular to the cell orientation. In embodiments, the extracellular matrix proteins may be laminin, collagen IV, and/or fibronectin.

Other embodiments relate to endothelial colony forming cells on polymer microfibers that deposit extracellular matrix proteins. The extracellular matrix proteins include, for example, laminin, collagen IV, and fibronectin. The extracellular matrix proteins wrap around the polymer microfiber, perpendicular to the cell orientation, along the fiber's circumference. Further, the extracellular matrix proteins can be above, among, or below the endothelial cells (between the cells and the microfiber).

Embodiments of the invention relate to the polymer microfibers cultured with endothelial progenitor cells being further seeded with a second cell type, such as mural cells (perivascular cells). Preferably, the mural cells are vascular smooth muscle cells or pericytes. In one embodiment, the vascular smooth muscle cells deposit ECM proteins, for example collagen type I, collagen type III and/or elastin. Embodiments include the ECM proteins, for example collagen types I and III, and. elastin, located beneath the vascular smooth muscle cell layer and above the endothelial progenitor cell layer. In another embodiment, pericytes deposit ECM proteins, for example collagen types III and IV. The collagen type IV is located above the endothelial progenitor cell layer.

Other embodiments of the invention are microvascular structures including an electrostretched polymer microfiber seeded with endothelial progenitor cells. Endothelial progenitor cells, such as endothelial colony forming cells, adhere to the surface of the polymer microfiber, align longitudinally with the microfiber, and deposit extracellular matrix circumferentially organized. Extracellular matrix proteins, including for example, laminin, collagen IV, and fibronectin, can encircle the polymer microfiber. The extracellular matrix proteins are oriented perpendicular to the cell orientation, along the fiber's circumference. Further, the extracellular matrix proteins are above, below, or among the endothelial cells. The term “below the endothelial cells” means that the ECM protein is between the cells and the microfiber. In some embodiments the microvascular structure comprising an electrostretched polymer microfiber seeded with endothelial progenitor cells is further seeded with a second cell type, for example mural cells. Preferably, the mural cells are vascular smooth muscle cells or pericytes. In some embodiments, the vascular smooth muscle cells deposits ECM proteins, for example collagens type I and type III, fibronectin, laminin, and elastin. Embodiments include the ECM proteins, for example collagen type I, type III and elastin located beneath the vascular smooth muscle cell layer and above the endothelial progenitor cell layer. In other embodiments, pericytes deposit ECM proteins, for example collagen types I, III and IV, fibronectin and laminin. The collagen type IV is located above the endothelial progenitor cell layer.

In further embodiments of the invention, the polymer microfiber induces increased deposition of ECM proteins by endothelial progenitor cells compared to two-dimensional cultures. These ECM proteins include for example, fibronectin, laminin, and/or collagen IV. The polymer microfibers also induce increased deposition of ECM proteins by perivascular cells seeded on the microfiber. The perivascular cells preferably are vascular smooth muscle cells and/or pericytes. The perivascular cells deposit increased ECM proteins including for example, collagens type I, III and IV, fibronectin, laminin or elastin. In embodiments, the increased ECM proteins deposited include fibronectin, laminin, elastin, and collagens type I, III, and IV.

Another embodiment includes a luminal multicellular microvascular structure. The luminal multicellular microvascular structure is formed from an electrostretched polymer microfiber seeded with endothelial progenitor cells and a second cell type, for example perivascular (mural) cells, wherein the polymer microfiber is degraded after attachment of the cells and deposition of the extracellular matrix. The polymer microfiber may be degraded with an enzyme, such as plasmin. Preferably, the endothelial progenitor cell is an endothelial colony forming cell, and the perivascular cell is a vascular smooth muscle cell or pericyte. In another embodiment, the luminal multicellular microvascular structure is hollow.

In the embodiments of the invention, the polymer is selected from alginate, fibrin (fibrinogen), gelatin, hyaluronic acid, or combinations thereof. In particular embodiments, the polymer is fibrin. In the embodiments, endothelial progenitor cells are endothelial colony forming cells, and the perivascular cells are vascular smooth muscle cells or pericytes. Endothelial colony forming cells deposit ECM proteins such as fibronectin, laminin, or collagen type IV. Vascular smooth muscle cells deposit ECM proteins such as collagen types I, III, IV, laminin, elastin and fibronectin. Pericytes deposit ECM proteins such as collagen types I, III, IV, laminin, and fibronectin.

Embodiments of the invention include a method of sequentially controlling microvascular vessel formation comprising the steps of preparing a tubular polymeric scaffold, for example a polymeric hydrogel microfiber comprising electrostretched polymer microfibers; seeding the microfiber with endothelial progenitor cells; co-culturing the endothelial cell-seeded microfiber with a second cell type such as perivascular (mural) cells, and varying the growth factors used for each step. The endothelial cells deposit extracellular matrix that produces extracellular proteins that encircle the microfiber, and are oriented perpendicular to the cell orientation, along the fiber's circumference, wherein formation of microvasculature vessel is sequentially controlled. In a preferred embodiment, the polymer is alginate, fibrin (fibrinogen), gelatin, hyaluronic acid, or combinations thereof. In a particular embodiment, the polymer is fibrin. In a preferred embodiment, the endothelial progenitor cells are endothelial colony forming cells and the perivascular cells are vascular smooth muscle cells or pericytes. In another embodiment, the method further comprises degrading the microfiber after seeding and culture of cells and deposition of extracellular matrix. Degrading the microfiber may be with an enzyme, such as plasmin.

Another embodiment includes a system for sequentially controlling microvascular vessel formation comprising a tubular polymeric microfiber comprising electrostretched polymer microfibers for forming a matrix for the culture of cells that form the vasculature; endothelial progenitor cells seeded on the polymer microfiber for forming a vascular lining; and perivascular (mural) cells co-cultured with the endothelial cell-seeded microfiber for forming a mural cell layer, and with endothelial progenitor cells depositing extracellular matrix proteins that encircle the microfiber and are oriented perpendicular to the cell orientation along the fiber circumference, wherein a microvascular structure is formed. In an embodiment, the polymer is alginate, fibrin (fibrinogen), gelatin, hyaluronic acid, or combinations thereof. In a particular embodiment, the polymer is fibrin. In a preferred embodiment, the endothelial progenitor cells are endothelial colony forming cells and the perivascular cells are vascular smooth muscle cells or pericytes.

### BRIEF DESCRIPTION OF THE DRAWINGS

FIG. 1 ECFCs attached and aligned on fibrin hydrogel microfibers. (A) Schematic of experimental procedure including electrostretching, ECFC seeding, and tumbling. Drawing not to scale. Scanning electron microscopy (SEM) of critical point-dried fibrin microfiber showing aligned topography on the microfiber surface. Scale bar is 10 μm. (B-D) Confocal Z-stack image reconstructions of ECFCs seeded on fibrin hydrogel microfibers horizontally aligned after 5 days in culture; F-actin (phalloidin staining) is shown in green, EC-specific markers (VECad, CD31, or vWF) in red, and nuclei in blue. Yellow arrows indicate the direction of stretching and nanotopography on microfiber surface. Scale bars are 50 μm. n≧2 per stain with quadruplicates. (E) Confocal Z-stack image reconstructions of ECFCs seeded on fibrin microfibers after one day in culture; F-actin (phalloidin staining) are shown in green, CD31 in red, and nuclei are counterstained in blue. Scale bars are 100 μm.

FIG. 2 ECFCs deposit ECM circumferentially on fibrin hydrogel microfibers. Confocal stack image reconstructions of ECFCs on fibrin microfibers after (A) 1 and (B) 5 days in culture. Scale bars are 200 μm. (C) High magnification confocal images of laminin, fibronectin and collagen IV wrapping around the fibrin microfibers. (D) TEM images of cross-sectional slices of a cell-fibrin microfiber construct after 5 days in culture (i-ii) with cells and (iii) without cells. (ii) is a higher magnification image for the boxed area in (i). F=Fibrin; E=ECM; C=Cells. (E) Cross-sectional projections of confocal Z-stack images of ECFCs on fibrin microfibers after 5 days. F-actin (phalloidin) is shown in green, ECM proteins (collagen IV, laminin, fibronectin) in red or magenta, and nuclei in blue. Yellow arrows indicate the direction of nanotopography on microfiber surface. a-n≧2, b-e n≧5 per stain with quadruplicates. (F) and (G) High magnification confocal Z-stack image reconstructions of ECFCs-seeded fibrin microfibers after 5 days in culture showing (F) wrapping ribbon-like organization of Collagen IV, laminin and fibronectin (in red; Scale bars are 50 μm) and (G) horizontal orientation of ECFCs with circumferential organization of the deposited Collagen IV (red). Yellow arrow indicates the direction of nanotopography. Scale bars are 100 μm.

FIG. 3 Nanotopography and geometry differently effect ECM organization. Confocal Z-stack image reconstructions of (A) ECFCs on 2D fibrin sheets after 5 days in culture. Yellow arrows indicate the direction of nanotopography. Scale bars are 50 μm. n=2 with duplicates. (B) ECFCs on PES 3D fibrin-coated fibers with random non-aligned topography after 5 days in culture. Scale bars are 100 μm. n>4 with quadruplicates. Actin filaments (phalloidin) are shown in green, ECM proteins (collagen IV, laminin, fibronectin) in red, and nuclei in blue. Box-and-whisker plots showing ECFCs (C) and ECM (D) angle of orientation on PES and fibrin hydrogel microfibers after 5 days in culture. (E) Standard deviation of ECM angle of orientation. Error bars represent 5-95% confidence intervals. Significance levels in the mean represented by ***p<0.001. n≧2 with quadruplicates. (F) SEM of critical-point dried fibrin fiber sheets showing aligned topography on the surface. Scale bar is 2 μm. (G) SEM of critical-point dried PES microfibers (i) coated with fibrin showing random topography and (ii) uncoated showing smooth topography. Scale bars are 10 μm.

FIG. 4 Disrupting actin and microtubule organization does not affect ECM organization. Confocal Z-stack image reconstructions of ECFCs seeded on fibrin microfibers for 24 hrs followed by treatment with (A) cytochalasin D or (B) nocodazole for 24 hrs and 48 hrs in culture. (C) Low (left) and high (right) magnification of ECFCs seeded on fibrin microfibers for 72 hrs without drug treatment, serving as control. F-actin (phalloidin staining) is shown in green, microtubules (a-tubulin) in red, ECM proteins (collagen IV or fibronectin) in red or magenta, and nuclei in blue. Yellow arrows indicate the direction of nanotopography. Scale bars are 100 μm except of high magnification in (C) that is 50 μm. (D) Box-and-whisker plots and (E) standard deviation of ECM angle of orientation. Error bars represent 5-95% confidence intervals. n=2 with quadruplicates. (F)-(H) Confocal Z-stack image reconstructions of ECFCs seeded on (F) fibrin microfibers or (G) on Petri dish for 24 h followed by treatment with cytochalasin D for 48 h in culture. (H) ECFCs seeded on fibrin microfibers and treated immediately with cytochalasin D for 72 hrs of culture. F-Actin filaments (phalloidin) are shown in green, collagen IV in red, fibronectin or laminin in magenta, and nuclei in blue. Yellow arrows indicate the direction of nanotopography on fibrin microfibers. Scale bars are 50 μm in (F)-(G) and 100 μm in (H). (I)-(K) Confocal Z-stack high-magnification image reconstructions of ECFCs seeded on (I) fibrin microfibers or (J) Petri-dishes for 24 h followed by treatment with nocodazole for 48 h of culture. (K) ECFCs seeded on fibrin microfibers and treated immediately with nocodazole for 72 hrs of culture. F-Actin filaments (phalloidin) in green, microtubules (a-tubulin) in red, Collagen IV in magenta, and nuclei in blue. Yellow arrows indicate the direction of nanotopography on fibrin microfibers. Scale bars are 50 μm in (I) (left), (J) (right) and (K) (right); 20 μm in (I) (right); and 100 μm in (K) (left). (L)-(N) Confocal Z-stack image reconstructions at different magnifications of ECFCs-seeded fibrin microfibers after 3 days in culture showing non-confluent ECFCs with circumferential organization of the deposited Collagen IV (red). Yellow arrow indicates the direction of nanotopography. Scale bars are (L) 200 μm, (M) 100 μm, (N) 50 μm.

FIG. 5 Microfiber curvature influences ECM organization. (A) Confocal Z-stack image reconstructions of Collagen IV deposition on fibrin microfibers with different diameters. Scale bars are 200 μm (B) Scatter plot and (C) standard deviation of ECM angle of orientation on microfibers with different diameters. Error bars represent 5-95% confidence intervals. Significance levels in the distribution represented by ***p<0.001. n=2 with quadruplicates. (D)-(F) High magnification confocal Z-stack image reconstructions of ECFCs-seeded fibrin microfibers with different diameter after 5 days in culture showing Collagen IV in red. Scale bars are 50 μm. Yellow arrow indicates the direction of nanotopography.

FIG. 6 Co-cultured vSMCs deposit new ECM, Confocal Z-stack image reconstructions of fibrin microfibers seeded with ECFCs followed by (A) co-culture of vSMCs for 2 days. n=2 with quadruplicates. Scale bars are 200 μm. Co-cultured vSMCs for 3 days showing (B) wrapping and (C) aligned arrangement. Scale bars are 100 μm. (D) Collagen Type I deposited by co-cultured vSMCs after 3 days in co-culture. Scale bars are 100 μm. (E) Cross-sectional projection of confocal Z-stack images of vSMCs after 5 days in co-culture. Arrowheads indicate SM22⁻ cells. Scale bars are 50 μm. (B)-(E) n>3 with quadruplicates. (F) Confocal Z-stack image reconstruction and (G) cross-sectional projection of co-cultured vSMCs after 5 days in co-culture. n=2 with quadruplicates. Scale bars are 50 μm. SM22 is shown in green, CD31 in red, collagen I and elastin in red, and nuclei in blue.

FIG. 7 Co-cultured Pericytes proliferate and deposit new ECM. Confocal Z-stack image reconstructions of fibrin microfibers seeded with ECFCs and cultured for 5 days followed by co-culture of pericytes for 10 days in ECFC media supplemented with 30 mM aminocaproic acid. (A) Pericytes express SM22 (red) and are located above ECFCs expressing lectin (green). Co-cultured pericytes showing (B) wrapping and (C) aligned arrangement. SM22 shown in green. (D) Projection and (E) cross-sectional projection of confocal Z-stack images of Collagen IV (green) deposited by co-cultured pericytes marked with smooth muscle actin (SMA) (red). Nuclei shown in blue. Arrowheads indicate ECFCs which are SM22⁻ and SMA⁻ (SMA negative) cells. Scale bars are 100 μm in (A) and (C), 50 μm in (B), (D), and (E).

FIG. 8 Deposition of Collagen IV (Col IV), fibronectin (Fn), and laminin (Lmn) by ECFCs in 3D vs. 2D. Confocal Z-stack image projections of ECM in (A) 3D fibrin microfibers and (B) 2D fibrin coated surfaces. Red: corresponding ECM; blue: nuclei, green: F-actin. Scale bars=100 μm. Insert scale bars=25 μm. (C) RT-PCR analysis of expression of ECM genes by ECFCs cultured on 2D vs. 3D. Error bars represent SEM. Significance levels in the distribution represented by *p<0.05 and **p<0.01. n>3.

FIG. 9 Deposition of Col I, III, IV, Fn, and Lmn by pericytes in 3D vs 2D. Confocal Z-stack image projections of ECM in (A) 3D fibrin microfibers and (B) 2D fibrin coated surfaces. Red: corresponding ECM; blue: nuclei, green: F-actin. Scale bars=100 μm. Insert scale bars=25 μm. Arrows, arrowheads, and double-headed arrows point to randomly deposited, non-polymerized, and aligned ECM proteins, respectively. (C) RT-PCR analysis of expression of ECM genes by vSMCs cultured on 2D vs 3D. (D) Orthogonal view of pericytes grown on microfibers. Arrowheads point to cells underneath outer cell layer, arrow points to extracellular deposited Lmn. Scale bar=20 μm. Error bars represent SEM. Significance levels in the distribution represented by *p<0.05 and **p<0.01. n>3.

FIG. 10 Deposition of Col I, III, IV, Eln, Fn, and Lmn by vSMCs in 3D vs 2D. Confocal Z-stack image reconstructions of ECM in (A) 3D fibrin fibers and (B) 2D fibrin coated surfaces. Red: corresponding ECM; blue: nuclei, green: F-actin. Scale bars=100 μm. Insert scale bars=25 μm. Arrows, arrowheads, and double-headed arrows point to randomly deposited, intracellular, and aligned ECM proteins, respectively. (C) RT-PCR analysis of expression of ECM genes by pericytes cultured on 2D vs 3D. ND=not determined. (D) Orthogonal view of pericytes grown on microfibers. Arrowheads point to cells underneath outer cell layer, arrow points to extracellular deposited Lmn. Scale bar=20 μm. Error bars represent SEM. n>2.

FIG. 11 Fiber degradation and viability of cells and ECM after plasmin treatment. (A) Light microscopy images of plasmin degrading fibrin microfibers in a concentration dependent manner. Scale bars=100 μm. (B) Immunofluorescence images of viability assays of ECFCs after 12 and 24 hr treatments with plasmin. Red: dead cells; green: live cells Scale bars=500 μm. (C) Confocal Z-stack image projections of fibrin fibers with ECFCs for 5 days and treated with plasmin for (I) 12 and (II) 24 hrs. (I) Col IV (red) Fn (green) Scale bars=200 μm, insert scale bars=100 μm. (II) Lmn (green) Left panel scale bar=200 μm, right panel scale bar=50 μm. Insert: Cross-sectional image, scale bar=50 μm. n>2.

FIG. 12 ECFC and perivascular cell co-cultures on fibrin microfibers. Confocal Z-stack image projections of ECFCs grown for 5 days on fibrin microfibers followed by 5 days more of (A) pericyte co-culture. Red: (I) VEcad (II) SM22 (III) Col III; blue: nuclei, green: F-actin. (B) vSMC co-culture. Red: (I) VEcad (II) SM22 (III) Eln; blue: nuclei, green: F-actin. Confocal Z-stack image 3D reconstructions of structures cultures for 5 days with ECFC followed by 5 more days with perivascular cells and then treated for 12 hrs with 0.25 CU/mL plasmin. (C) Pericyte co-culture. Red: (I) and (III) VEcad, (II) and (IV) Col III; magenta: (I) and (III) SM22; blue: nuclei; green: (I) and (III) F-actin, (II) and (IV) Col IV. (D) vSMC co-culture. Red: (I) and (III) VEcad, (II) and (IV) Eln; magenta: (I) and (III) SM22, (II) and (IV) Fn; blue: nuclei; green: F-actin. Scale bars=50 μm. n>2.

### DETAILED DESCRIPTION OF THE INVENTION

A novel in vitro model and system that recapitulates key aspects in the cellular and extracellular matrix (ECM) organization of the microvasculature is established in accordance with the invention. This model and system guide the formation of organized microvascular structures, induction of endothelial cell alignment and elongation, and demonstrates circumferential deposition of ECM proteins by endothelial progenitor cells, for example endothelial colony forming cells. The model reveals the role of vessel diameter on ECM organization during human microvascular growth. The model supports a step-wise vascular formation process via introduction of perivascular cells and different growth factors at varying time points, a current challenge in microvascular tissue engineering. A multicellular microvascular structure with an organized endothelium and multicellular perivascular tunica media is also disclosed.

Most current approaches for the in vitro study of the microvasculature in a 3D setting use hydrogels and scaffolds embedded with vascular cells, which are subject to spontaneous capillary bed formation (Hielscher A C, et al., Am J Physiology—Cell Physiology, 2012; 302: C1243-C1256; Soucy P A and Romer L H, Matrix Biology, 2009; 28: 273-283; Hanjaya-Putra D, et al., Blood, 2011; 118: 804-815, Moon J J, et al., Biomaterials, 2010; 31: 3840-3847; Pham Q P, et al., Tissue Eng, 2006; 12: 1197-1211; Benjamin L E, et al., Development, 1998; 125: 1591-1598; Gerhardt H, Betsholtz C, Cell and Tissue Research, 2003; 314: 15-23; Stratman A N, et al, Blood, 2009; 114: 5091-5101; Wang Z Z, et al., Nat Biotech, 2007; 25: 317-318; Koike N, et al., Nature, 2004; 428: 138-139; Melero-Martin J M, et al., Circulation Research, 2008; 103: 194-202; Levenberg S, et al., Nature Biotechnology, 2005; 23: 879-884; Hielscher A C, Gerecht S, Cancer Research, 2012; 72: 6089-6096; Soucy P A, et al., Acta Biomaterialia, 2011; 7: 96-105; Hanjaya-Putra D, et al., Biomaterials, 2012; 33: 6123-6131; Leslie-Barbick J E, et al., Biomaterials, 2011; 32: 5782-5789), or use micropatterned hydrogels to generate organized microvasculature structures (Baranski J D, et al., Proc Natl Acad Sci USA, 2013; Miller J S, et al., Nat Mater, 2012; 11: 768-774; Zheng Y, et al., Proc Natl Acad Sci USA, 2012; 109: 9342-9347). While these approaches are instrumental for studying angiogenic processes, they provide only partial control over the topographical cues presented to the cells by the ECM and possess limited opportunities to create and investigate multi-cellular vascular structures with proper ECM organization.

Previous studies in the field of angiogenesis, vasculogenesis, and vascular tissue engineering have either focused on studying the formation of capillaries and vessels with diameters below 100 μm (Hanjaya-Putra, D., et al., J Cellular and Molecular Medicine, 2010; 14(10):2436-2447; Hanjaya-Putra, D., et al., Blood, 2011; 118:804-815; Davis, G. E., et al., Birth Defects Research Part C: Embryo Today: Reviews, 2007; 81(4):270-285; Montano, I., et al., Tissue Engineering Part A, 2009. 16(1): p. 269-282; Chen, X., et al., Tissue Engineering Part A, 2008. 15(6): p. 1363-1371; Kobayashi, A., et al., Biochemical and Biophysical Research Communications, 2007. 358(3): p. 692-697; Dickinson, L. E., et al., Soft Matter, 2010. 6(20): p. 5109-5119; Tsuda, Y., et al., Biomaterials, 2007. 28(33): p. 4939-4946; Baranski, J. D., et al., PNAS, 2013. 110(19): p. 7586-7591), or have instead aimed to develop large-diameter tissue engineered vessels, most over 3 mm in diameter (Vaz, C. M., et al., Acta Biomaterialia, 2005. 1(5): p. 575-582; Kelm, J. M., et al., J Biotechnology, 2010. 148(1): p. 46-55; L'heureux, N., et al., FASEB Journal, 1998. 12(1): p. 47-56; Dahl, S. L., et al., Cell transplantation, 2003. 12(6): p. 659-666; Quint, C., et al., PNAS, 2011. 108(22): p. 9214-9219).

The invention overcomes challenges to developing physiologically relevant microvascular structures with diameters between 100 μm and 1 mm. It previously has been established that ECs can create vascular networks in vitro when cultured in 3D matrices such as hydrogels, yet these networks result in capillary beds with relatively small lumen diameters (Hanjaya-Putra, D., et al., J Cellular and Molecular Medicine, 2010; 14(10):2436-2447; Hanjaya-Putra, D., et al., Blood, 2011; 118:804-815; Davis, G. E., et al., Birth Defects Research Part C: Embryo Today: Reviews, 2007; 81(4):270-285; Montano, I., et al., Tissue Engineering Part A, 2009. 16(1): p. 269-282; Chen, X., et al., Tissue Engineering Part A, 2008. 15(6): p. 1363-1371). Therefore, to create vessels with a larger diameter the present invention guides the formation of cylindrical vascular structures in the order of hundreds of microns. Microfluidic channels have been used extensively to study microvascular development processes, yet most of these studies are done in channels with a square cross-section or in non-implantable devices (Verbridge, S. S., et al., J Biomedical Materials Research Part A, 2013. 101(10):2948-2956; Abaci, H. E., et al., Sci. Rep., 2014:4).

Recent studies have developed 3D microfluidic channel arrays with rectangular (Zheng, Y., et al., PNAS, 2012; 109(24):9342-9347) or circular cross-sections embedded in hydrogels (Miller, J. S., et al., Nature Materials, 2012; 11(9):768-774; Wu, W., et al., Advanced Materials, 2011; 23(24):H178-H183; Kolesky, D. B., et al., Advanced Materials, 2014; 26(19):3124-3130; He, J., et al., Adv. Healthcare Materials, 2013; 2(8): p. 1108-1113). However, these present limited success for constructing a multilayered structure with a continuous endothelium and a robust supporting multicellular mural cell layer. To address the need for mural cell involvement, some of these studies incorporated perivascular cells in the hydrogels encompassing the microfluidic channels, and afterwards seeded ECs in the lumen (Zheng, Y., et al., PNAS, 2012; Miller, J. S., et al., Nature Materials, 2012). While this approach allows the study of cell recruitment, it imposes a barrier for full investment of perivascular cells, which have to migrate through the hydrogels to find the developing microvessels. As such, perivascular cells in these systems were not demonstrated to form a uniform multicellular layer on top of the endothelium. Furthermore, these systems impart a barrier for the detailed study of ECM organization and EC-mural cell interactions due to chemical and physical limitations presented the hydrogels.

The microfiber according to the invention provides control over the topographical cues presented to cells by the ECM. The microfiber of the invention also presents opportunities to create and investigate multi-cellular vascular structures with proper ECM organization. The microfiber according to the invention has a nanotopography that induces longitudinal adhesion and alignment of endothelial progenitor cells, for example, endothelial colony-forming cells (ECFCs). The endothelial progenitor cells, such as ECFCs, deposit circumferentially organized ECM. The ECM wraps around the microfibers of the invention, which is independent of ECFCs' actin and microtubule organization. As established by the present invention, ECM encircling or wrapping around the microfibers is dependent on the curvature of the microfiber. According to the invention, microfibers with small diameters, for example less than about 500 μm, preferably about 100 μm to about 450, more preferably about 100 μm to about 370 μm, guide circumferential ECM deposition. Microfibers with larger diameters, 445 μm and higher, do not support wrapping ECM as effectively. The invention provides a novel in vitro structure and method for the sequential control of microvasculature development and reveals the unprecedented role of the endothelium in organized ECM deposition regulated by the microfiber curvature.

Unless defined otherwise, technical and scientific terms used herein have the same meaning as commonly understood by one of ordinary skill in the art to which this invention belongs. As used herein, the terms “perivascular cells” and “mural cells” have the same meaning. The term “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The present invention can be used as a model of the microvasculature that recapitulates both cellular and ECM organization, towards the understanding of microvasculature development and utilization of the model for regenerative medicine applications. Towards this, electrostretched microfibers designed to generate a micro-cylindrical mold with a line-grating nanotopography are used to enable both endothelial layer organization and co-culture of supporting perivascular (mural) cells, such as vascular smooth muscle cells (vSMCs) or pericytes. Microfibers used have diameters of about 500 μm or less, preferably ranging from about 100 μm to about 450 μm, corresponding to a poorly studied range of vasculature in the body, namely venules and arterioles. Furthermore, in the existing models of microvasculature, the deposition and organization of ECM proteins by endothelial cells has not been studied. Moreover, the full investment of mural cells on the endothelium of microvascular models has been challenging, due in part to the use of endothelial-lined void spaces in most models (Miller J S, et al., Nat Mater, 2012; 11: 768-774; Zheng Y, et al., PNAS USA, 2012; 109: 9342-9347), which introduces a cell migration barrier for mural cell investment. In contrast, the model of the invention allows not only high resolution studies of both cell and ECM organization; it allows introduction of mural cells after endothelial layer formation and enables full investment of these mural cells, recreating the media layer of microvasculature.

This new approach to create aligned hydrogel microfibers as described herein uses an electrostretching process from various polymer materials. Unique characteristics of the electrostretched polymer fibers are the internal and topographical alignment of the fibrous structure, generated as a result of both electrical field and mechanical shear-induced polymer chain alignment. Furthermore, the microfiber diameter is controllable and uniform as a result of the bundling and processing of the individual fibers composing the microfibers.

The typical electrospinning process, such as disclosed in International Publication WO2013/165975, incorporated by reference herein, consists of a syringe pump with a syringe containing a polymer solution of one or more polymers, a high voltage source, and a grounded collecting plate. The technique is based on inducing an electric charge on the polymer solution while applying an electric field between the syringe needle and the grounded collecting plate. As the solution is dispensed from the syringe, the electrostatic force opposes the surface tension of the polymer solution producing a Taylor cone. Eventually the electrostatic force overcomes the surface tension to produce a liquid jet stream. As the jet travels, the electric forces cause the stream to spin. At the same time, the solvent evaporates from the solution and the polymer fibers fall on the collecting plate forming an ultrathin nanofiber. (See WO2013/165975).

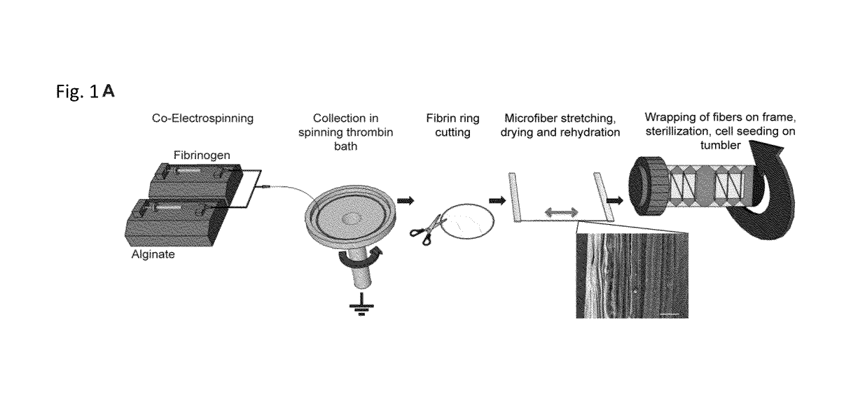

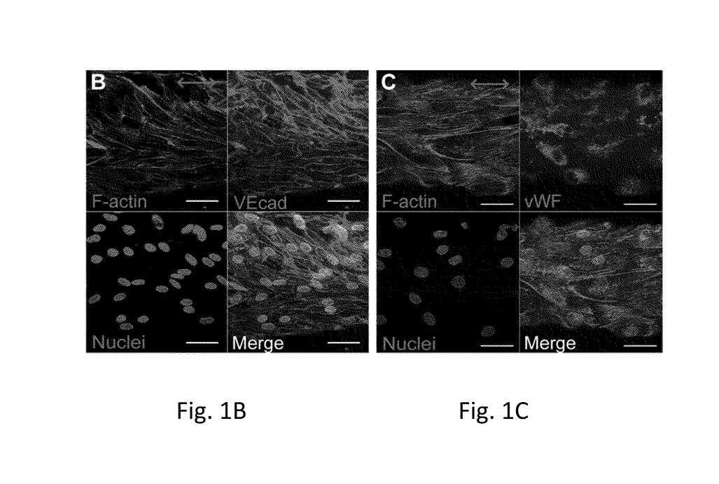

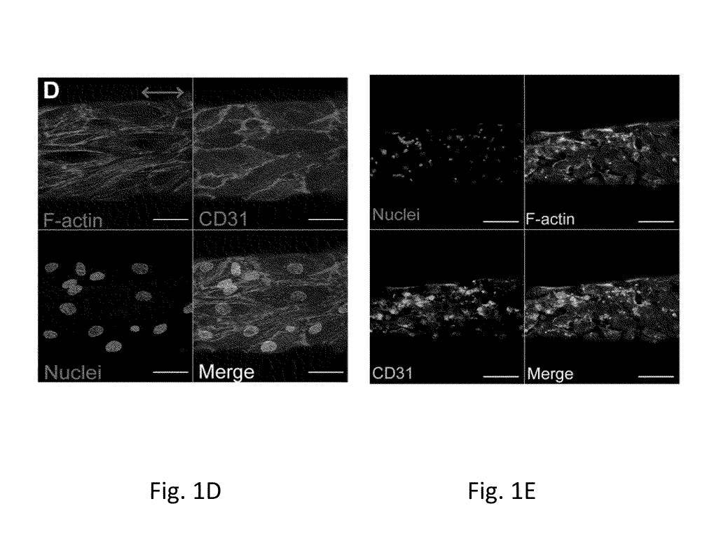

The novel electrospinning/electrostretching technique of the invention modifies the typical electrospinning processes, and produces hydrogel microfibers with a uniaxial aligned topography using a combination of electrical and mechanical stretching. The typical electrospinning process was modified so the collecting plate was a grounded rotating disc containing an aqueous solution (See FIG. 1). The polymer jet emitted from the syringe is deposited as nanofibers that fall on top of each other in the rotating disc bath. At this point, the bundle of aligned nanofibers is not cohesive. To make a cohesive microfiber, the electrospun nanofibers are collected together parallel to each other and stretched mechanically by the rotating disc, then air-dried so the nanofiber bundle becomes the microfiber. This bundle is further stretched to obtain a cohesive uniaxial internal and topographical alignment, which enhances the mechanical properties of the microfibers. Alternatively, the nanofibers can be collected together and partially stretched on a modified plastic frame to make a flat 2D nanofiber sheet.

Various polymers may be used in the electrostretching technique of the invention, such as natural polymers including alginate, fibrin (fibrinogen), gelatin, hyaluronic acid (HA), chitosan chondroitin sulfate, dextran sulfate, heparin, heparan sulfate, and functionalized derivatives thereof, and synthetic polymers selected from a polyester and a polyamide, such as polyacrylic acid derivatives and polyvinyl alcohol, including polylactic acid, poly(lactic-co-glycolic) acid, polyacrylate, poly(vinyl alcohol), poly(ethylene glycol), as well as combinations thereof, that produce hydrogel polymer fibers useful in the invention. Nanofibers formed from the polymer tray be crosslinked. Crosslinking may be achieved by any cross-linking method, including ionic crosslinking, ultraviolet crosslinking, enzymatic crosslinking, and chemical crosslinking reaction. For the electrostretching technique presented in the invention, the preferred polymers that can be used are alginate, gelatin, fibrin (fibrinogen), hyaluronic acid, and combinations thereof, with fibrin being the most preferred polymer.

Fibrin gels have been used to study microvasculature assembly (Dickinson L E, et al., Soft Matter, 2010; 6: 5109-5119; Bayless, K J, and Davis, G E, Biochemical and Biophysical Research Communications, 2003; 312: 903-913; Davis G E, and Bayless K J, Microcirculation, 2003; 10: 27-44; Bayless K J, et al., RGD-Dependent American Journal of Pathology, 2000; 156: 1673-1683; Dickinson L E, et al., Lab Chip, 2012; 12: 4244-4248), vSMC responses (Ahmann K A, et al., Tissue Eng Part A, 2010; 16: 3261-3270; Long J L, and Tranquillo R T, Matrix Biol, 2003; 22: 339-350) and multicellular organization (Lesman A, et al., Biomaterials, 2011; 32: 7856-7869). Fibrin is used as a matrix material for the microfiber according to the invention, including use in preparing hydrogel microfibers as a template for the step-wise creation of microvasculature of the invention.

The polymer microfiber of the invention has an aligned nanotopography that guides alignment and elongation of endothelial progenitor cells, such as ECFCs. The invention provides a microfiber having cylindrical shape and tunable diameter of the fibers, which are novel features in hydrogels that allow detailed 3D view and analysis of microvasculature development. The novel features also allow analysis of the effect of curvature of the fiber on cell processes. In an embodiment of the invention, fibrin is used for the hydrogel microfiber, which makes the scaffold not only biocompatible, bio-adhesive, and pro-angiogenic, but also easily degradable, such as through plasmin fibrinolysis. The development of delimited microvascular structures in small size range, for example less than about 500 μm, preferably about 100 μm to 450 μm, with demonstrated detailed cell and ECM organization has not been previously achieved.

According to the invention, aligned polymer microfiber (nanofiber bundles) used as a cylindrical platform control the organized adhesion of endothelial progenitor cells, such as ECFCs. In an embodiment of the invention, endothelial progenitor cells like ECFCs are cultured on electrospun fibrin microfibers that have a diameter of less than about 500 μm, preferably 100 μm to about 450 μm. The cells attach throughout the microfibers. ECFCs form a continuous monolayer over the entire microfiber, with a distinctive elongated and mature morphology. The polymer microfibers offer an innovative approach in which ECFCs are seeded on the surface of an electrostretched microfiber, as opposed to seeded in the body of a nanofiber mesh, as conventional electrospun scaffolds have been used (Pham Q P, et al., Tissue Eng, 2006; 12: 1197-1211; Kumbar S G, et al., Biomed Mater, 2008; 3: 034002; Christopherson G T, et al., Biomaterials, 2009; 30: 556-564; Chua K N, et al., Biomaterials, 2006; 27: 6043-6051).

In an embodiment of the invention, ECFCs cultured on polymer microfibers of the invention exhibit typical membrane expression of endothelial markers VEcad and CD31, and cytoplasmic expression of von Willebrand factor (vWF) (FIG. 1B-D) Expression of the endothelial markers demonstrates that fibrin microfibers support the adhesion and culture of ECFCs. This unique approach allows detailed control of the cellular assembly of microvasculature, as the fibrin microfibers present a blueprint with a unique aligned nanotopography for adhesion of ECFCs.

In embodiments of the invention, ECFCs deposit ECM that wraps circumferentially around the polymeric microfiber of the invention. Examining the ECFC-deposited ECM organization on the microfiber after 5 days in culture, ECFCs deposit ECM proteins, for example, collagen IV, fibronectin and laminin. ECM proteins wrap in discrete circumferentially aligned segments on the microfibers, perpendicular to the cells macroscopic cellular alignment and intracellular cytoskeletal organization. This feature of the ECFCs in which they deposit abundant ECM (Kusuma S, et al., FASEB J, 2012; 26: 4925-4936), that is assembled circumferentially on a micro-cylindrical platform, recognizes an active role of the endothelium in the construction of the extracellular components of the microvasculature. In longer period cultures of ECFCs on fibrin microfibers (i.e. >10 days) full coverage of the structures by ECM was observed (data not shown). However, at this time point the initial circumferential organization of the ECM could not be analyzed due to several layers of ECM being deposited on top of each other. For quantification purposes, cultures of ECFC were analyzed when ECM organization was evident, before full coverage was achieved.

Circumferential wrapping of ECM by ECFCs has not been observed previously. To elucidate whether the ECFC alignment or the cylindrical structure and 3D aspect of the scaffold had a direct effect on ECM organization, two different systems have been used. A flat polymer sheet with the same nanotopography as the polymer microfibers can be used as a first scaffold; the flat polymer sheet varies the shape and geometry of the scaffold. ECFCs align with the nanotopography of the polymer sheets, but the ECM is deposited with no distinguishable organization. A second scaffold for use is a polymer microfiber, such as polyethersulfone (PES), coated with a polymer such as fibrin, which maintains the microfiber's spatial geometry, but has a random surface topography. ECFCs seeded on the polymer microfiber, for example PES fibers, are not induced to align with the fiber's longitudinal axis, but they deposit ECM wrapping circumferentially around the polymeric microfiber, similar to ECFCs seeded on fibrin microfibers. Without being bound by theory, it is believed that the cylindrical shape of the fibers, and not the cellular organization induced by the scaffold's nanotopography, is necessary for ECM circumferential deposition.

The cytoskeleton is known to regulate endothelial alignment (Ranjan A, and Webster T, Nanotechnology, 2009; 20: 305102; Liliensiek S, et al., Biomaterials, 2010; 31: 5418-5426; Bettinger C J, et al., Adv Mater, 2008; 20: 99-103; Lu J, et al., Acta Biomater, 2008; 4: 192-201) and drive angiogenic responses through ECM-interactions (Bayless K J, Davis G E, Journal of Cell Science, 2002; 115: 1123-1136; Hanjaya-Putra D, et al., J Cell Mol Med, 2009). Cytoskeleton re-arrangement of ECFCs seeded on fibrin microfibers through actin and tubulin configuration revealed that ECFC alignment on the microfibers is not instrumental for the circumferential deposition of ECM by ECFCs Inhibition of neither actin filament nor microtubule polymerization affected ECM circumferential organization around the fibrin microfiber. Thus, ECM expression and organized deposition from ECFCs can be independent of ECFC cellular organization. Furthermore, shorter culture time periods, for example 3 days, which did not always produce a confluent endothelium still resulted in wrapping ECM, suggesting an independence of cell density on ECM organization.

Overall, even though aligned topography of the polymer microfibers induces ECFC alignment, the findings that either ECFCs seeded on PES fibers or ECFCs seeded on fibrin microfibers with disrupted actin and microtubule organization still produce wrapping ECM suggest that ECM organization is regulated by 3D geometric sensing of curvature, rather than by the nano-topography of the scaffold.

In an embodiment of the invention, the microfiber system demonstrated that circumferential wrapping of the ECM depends on the curvature of the microfibers. While curvature of nano-scale features of ECM has been suggested to regulate cellular responses (Vogel V, and Sheetz M, Nat Rev Mol Cell Biol, 2006; 7: 265-275), its effect on cellular responses at the micro-scale and during microvascular formation and organization has not been previously investigated. In this invention, polymer microfibers with an aligned nanotopography are generated by electrostretching. Microfibers are produced with uniform and tunable diameters while preserving the aligned nanotopography. In one exemplary embodiment, fibrinogen is mixed with alginate in-line and then charged with electric potential of about 2 kV to about 6 kV; the mixture is extruded through a 25-gauge needle. The fibrinogen-alginate solution jet is collected at a distance of about 3 to about 5 cm from the needle tip, in a grounded, rotating bath containing calcium chloride and thrombin as a cross-linking solution, to generate aligned nanofibers that can later be bundled to form microfibers with an aligned nanotopography. Microfibers are generated with different diameters by varying the collection time, such as from about 5 min and higher, preferably from any time point from about 5 min to about 80 min, more preferably from any time point from about 7 to about 80 min. Microfiber formation may include crosslinking nanofibers by any cross-linking method, including ionic crosslinking, ultraviolet crosslinking, enzymatic crosslinking, and chemical crosslinking reaction. After formation of crosslinked fibrin-alginate nanofibers, alginate is removed, preferably by soaking fibers in a sodium nitrate solution. Excess sodium citrate is washed off and the resulting fibrin nanofibers are collected as an aligned bundle, stretched preferably to 150% of their initial length, and dried. Resulting fibrin microfibers can be wrapped around a custom-made plastic frame and then sterilized for use.

Microfibers with diameter ranging from about 100-500 μm were examined. Microfibers of up to about 450 μm in diameter guide the organized wrapping of deposited ECM. Preferably, microfibers have a diameter about 100 μm to about 450 μm, more preferably about 100 μm to about 400 μm, about 100 to about 350 μm, about 100 to about 300 μm, about 100 to about 250 μm, about 200 μm, about 150 μm, and diameters within the ranges, such as about 110 μm, about 120 μm, 130 μm, 140 μm, 150 μm, 200 μm, 250 μm, 300 μm, 350 μm, 400 μm, 450 μm. Larger microfibers resulted in a more random ECM organization. This observation is the first to suggest an effect of the microtubular curvature on ECM organization. In contrast to studies using larger diameter templates to study ECM deposition of perivascular cells (Grassl E, et al., J Biomedical Materials Research Part A, 2003; 66: 550-561), the smaller diameter fibers demonstrate the role of curvature at the micro-scale.

The versatility of the polymer microfiber system is evident in allowing sequential and controlled introduction of other cells, which can deposit ECM. ECFCs will initially have an inverted polarity due to the presence of the fiber where the luminal surface would be and an absence of a tunica media on top. The first step towards correcting this inverted polarity was obtaining full investment of mural cells on top of the endothelium. Embodiments of the invention relate to the polymer microfiber cultured with endothelial progenitor cells being further seeded with a second cell type, such as perivascular cells, or mural cells. Preferably, the perivascular cell or mural cell is a vascular smooth muscle cell (vSMC) or pericyte. Both pericytes and vSMCs introduced independently into the model after ECFC culture on the fibrin microfibers were found to attach to the ECFC-seeded microfibers. The vSMCs and perictyes showed organizations that varied from the random, sporadic attachment typical of muscular venules, to the circumferential wrapping observed in arterioles under physiological conditions (Standring S, 2008). The organization, however, was in a multilayer configuration as opposed to the monolayer formed by the ECFCs, in accordance to the multilayer organization observed in the tunica media of native vessels. The different morphologies observed between vSMCs, pericytes and ECFCs are likely a result of lack of pulsatile flow, which has been shown to induce circumferential wrapping of vSMCs in different studies (Lee A A, et al., J Biomech Eng, 2002; 124: 37-43; Liu B, et al., Biophysical Journal, 2008; 94: 1497-1507). Indeed, the lack of internal pressure in this system is more conducive to the generation of muscular venules, which have much lower blood pressures compared to arterioles and contain randomly oriented vSMCs or pericytes (Standring S, 2008).

A further step to correct the polarity of the ECFCs in the model is obtaining a defined lumen to create a hollow microvascular vessel. A further advantage of the fibrin microfiber scaffold is its biodegradability; fibrin can be easily degraded in a controlled manner using plasmin in conditions that do not affect cell viability (Neidert M R, et al., Biomaterials, 2002; 23: 3717-3731). In an embodiment of the invention, degradation of the fibrin microfibers generates a hollow microstructure with a defined lumen, which can be used for applications in vivo. Thus, the fibrin microvascular system provides opportunities to correct the initial ECFC polarity and to study flow-induced vSMC or pericyte organization post fibrin core degradation.

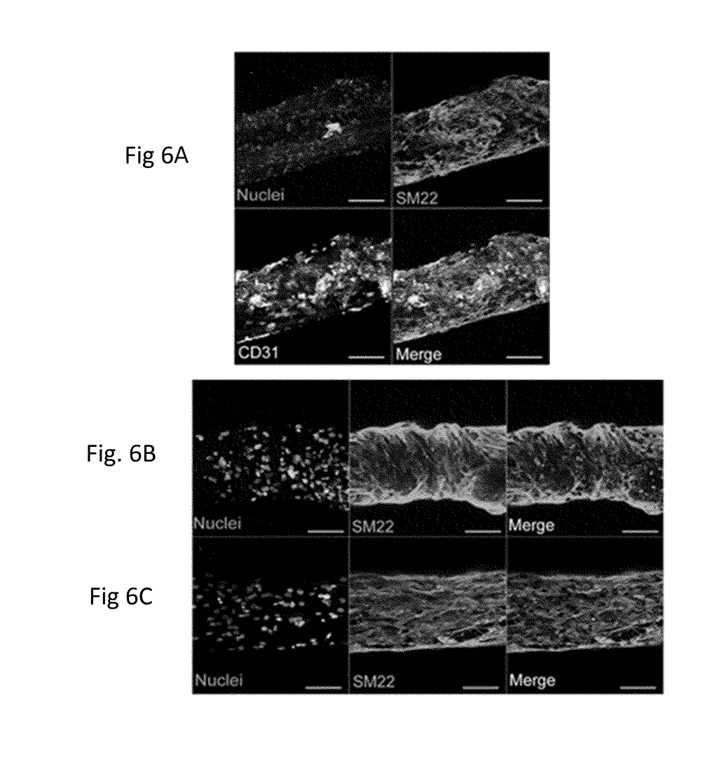

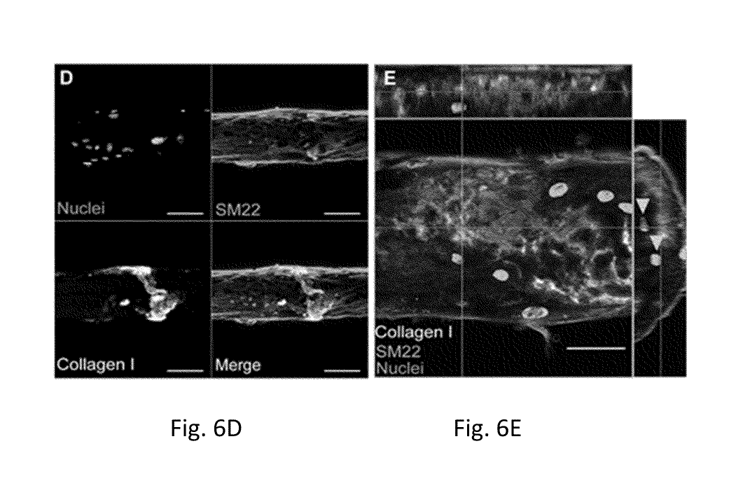

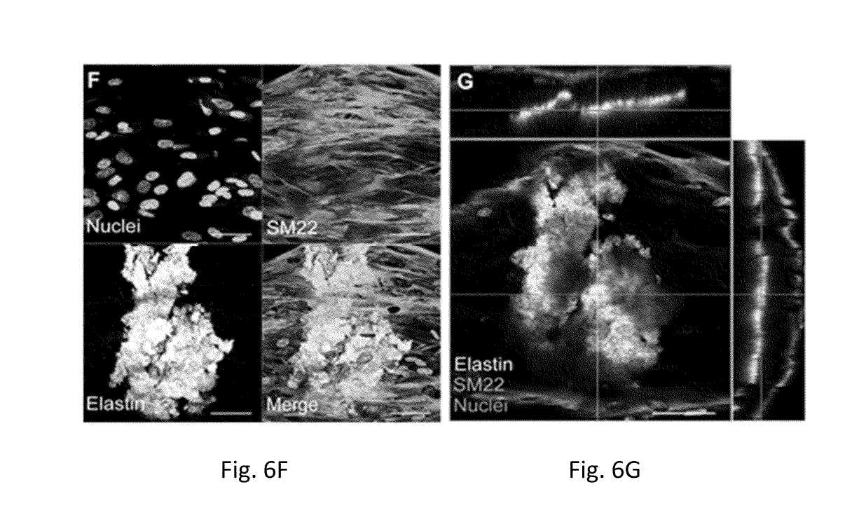

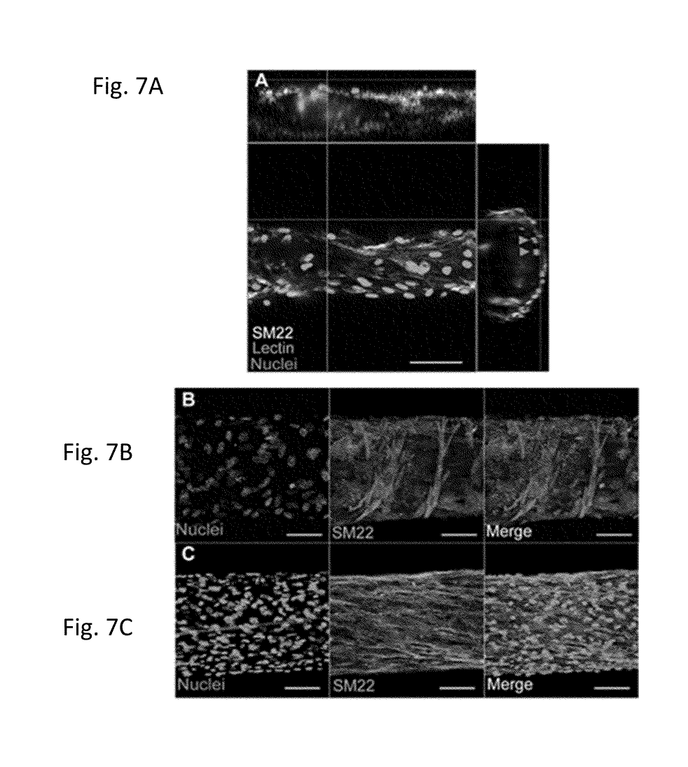

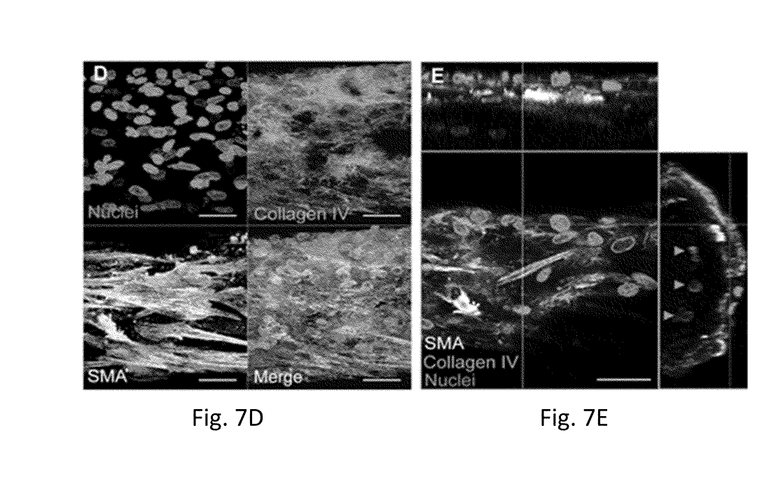

vSMCs and pericytes attach on ECFC-seeded microfibers and deposit extracellular proteins. vSMCs attach and grow on the ECFC layer (FIGS. 6A-C) and deposit collagen Type I and elastin (FIGS. 6D-G). The collagen type I and elastin are located below and in between the vSMC layer, and above the ECFCs (FIGS. 6E,G). Pericytes attach and grow on the hydrogel microfiber scaffold (FIGS. 7A-C), deposit collagen Type IV (FIGS. 7D-E), which is located below and in between the pericyte layer and above the ECFCs.

These results establish that co-cultured vSMCs and pericytes deposited ECM components that organize the subendothelial connective tissue and internal elastic lamina, located in between the endothelium and the tunica media, as well as components of the tunica media itself. Co-culture experiments in ECFC media was found to support ECFC, pericytes, and vSMC viability.

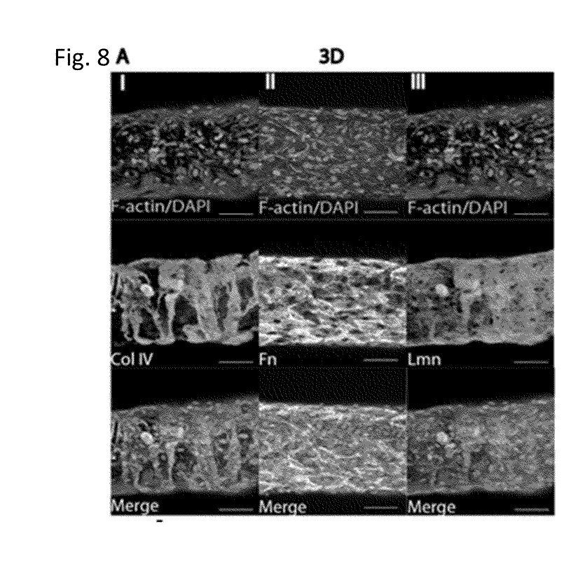

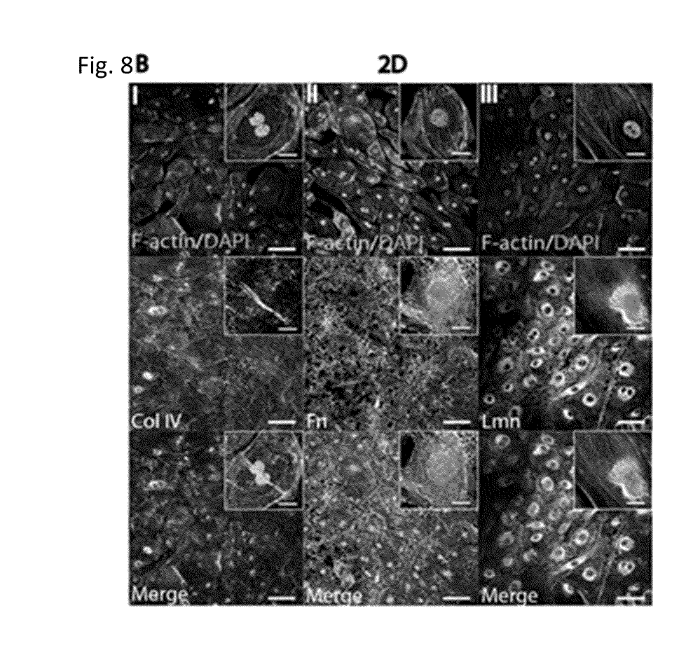

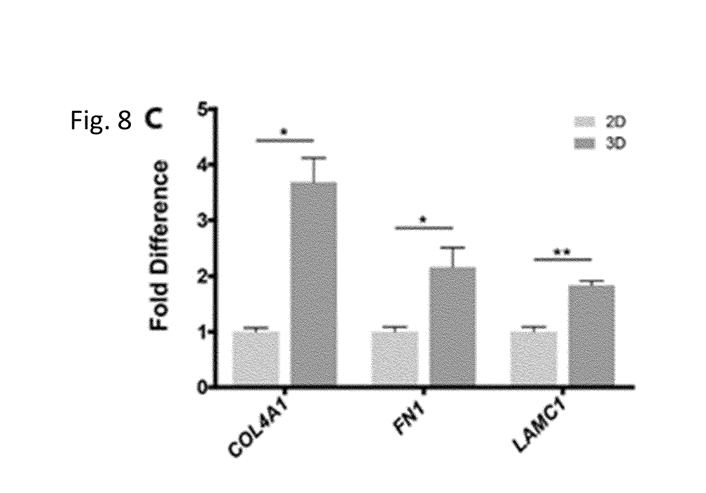

Self-supporting structures with the abundant vascular ECM deposition necessary to withstand pulsatile flow as well as vasoconstriction and vasodilatation are disclosed. ECM proteins such as collagens, laminin, and elastin have been shown to provide these biomechanical properties. To date, the study of ECM protein deposition by different vascular cells in 3D constructs either alone or in co-culture has been limited (Davis, G. E. and D. R. Senger, Circulation Research, 2005; 97(11): p. 1093-1107.). In an embodiment of the invention, increased quantities of ECM were deposited on the 3D microfibers of the invention compared to 2D cultures after seeding ECFCs, pericytes, and vSMCs in 2D and 3D culture. The 2D surfaces were coated with a thick fibrin hydrogel layer to provide a similar stiffness and bioactive substrate compared to its 3D counterpart. For ECFCs, immunofluorescence microscopy revealed increased deposition of ECM proteins Col IV, Fn, and Lmn in 3D compared to 2D (FIGS. 8A,B). This increased expression was found to be about 2-fold for Fn and Lmn and almost 4-fold for Col IV, as measured by RT-PCR (FIG. 8C). These results indicate the existence of a geometrical or biomechanical sensing pathway that upregulates the production of these proteins comprising the basal lamina of native endothelium (Laurie, G., et al., Cell Biology, 1982; 95(1): p. 340-344).

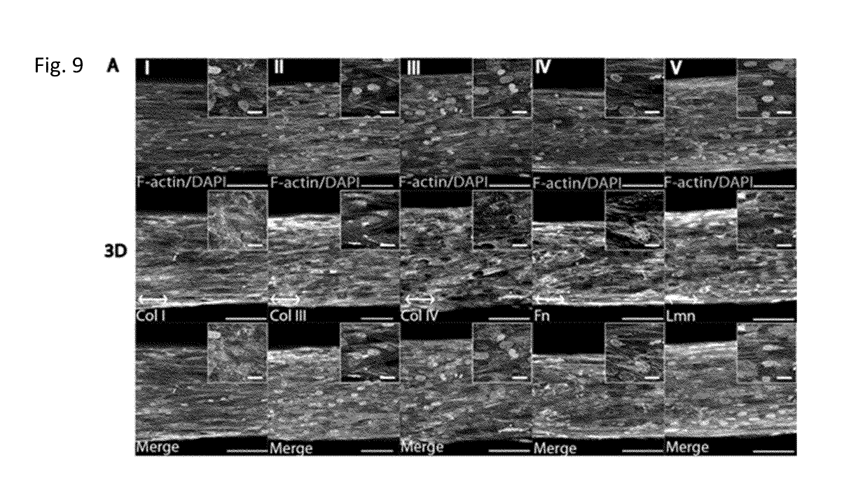

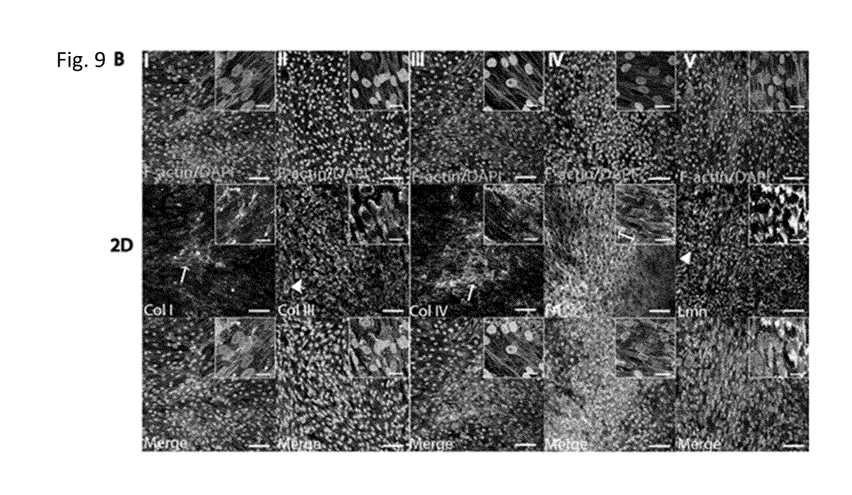

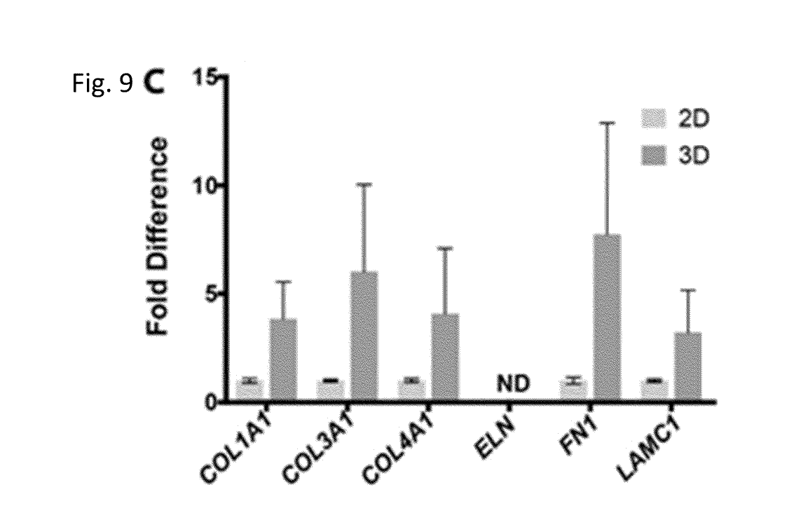

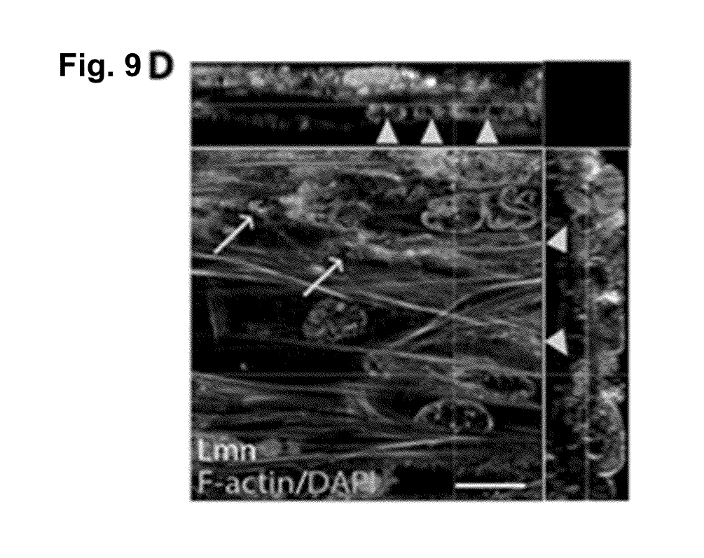

Similar studies performed on pericytes and vSMCs revealed these cell types produce several different ECM proteins that comprise the subendothelial connective tissue, internal elastic lamina, and ECM of the tunica media of blood vessels (Brooke, B. S., et al., Trends in Cell Biology, 2003; 13(1):51-56; Hungerford, J. E., et al., Developmental Biology, 1996; 178(2):375-392). In the fibrin hydrogel microfiber of the invention, both pericytes and vSMCs produced Col types I, III, IV, as well as Fn and Lmn. Additionally, only vSMCs produced Eln, which is in accordance to native vasculature where elastic tissue is found predominantly in arterioles and arteries with a full vSMC layer and not in smaller capillaries or venules invested only by pericytes (Hibbs, R. G., et al., Am Heart J, 1958. 56(5):662-670; Yen, A. and I. M. Braverman, J Investig Dermatol, 1976. 66(3):131-142; Brooke, B. S., et al., Trends in Cell Biology, 2003; 13(1):51-56). Notably, there is an increase in average expression of all ECM proteins deposited by perivascular cells in 3D compared to 2D microfibers, though the upregulation was only statistically significant for Col I, Col IV, and Eln deposition by vSMCs (FIGS. 9A-C, 10A-C). Furthermore, the morphological expression of these proteins was found to be more organized in 3D than 2D substrates, and Lmn was found to be deposited extracellularly in 3D microfiber substrates only. This is evidence of distinct metabolic pathways that regulate the expression and deposition of different ECM proteins by vSMCs and pericytes. The fibrin microfibers provide an appropriate microenvironment for abundant, organized ECM deposition.

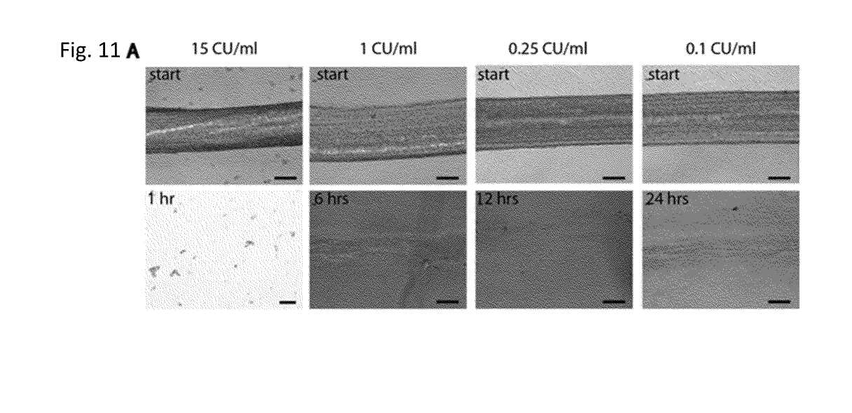

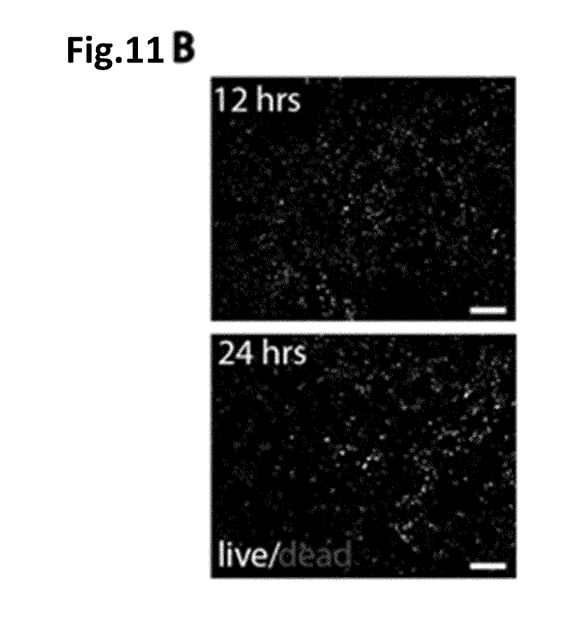

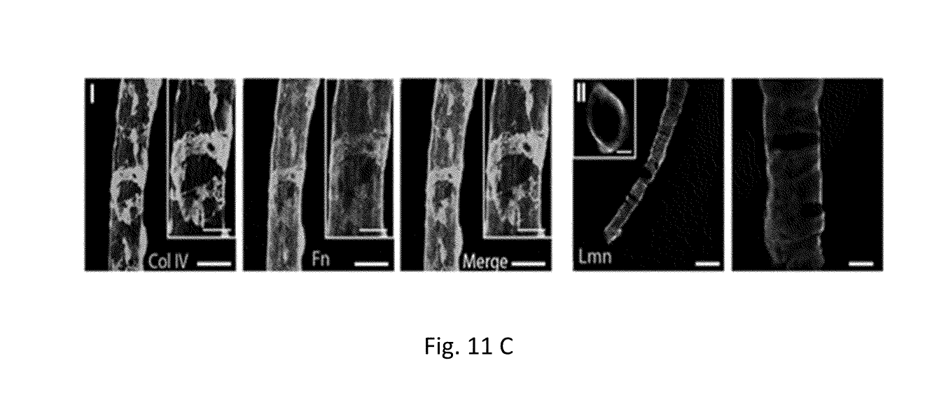

In a further embodiment, hollow microvascular vessels are prepared by removing the polymer microfiber core from the microvascular structures. An advantage of fibrin as a polymer biomaterial, besides its natural biocompatibility, bioadhesiveness, and angiogenic promoting characteristics (Clark, R. A. F., 2003; 121(5): p. xxi-xxii), is its biodegradability in response to enzymes such as plasmin, the enzyme responsible for eliminating fibrin blot clots in the human body (Rijken, D. C. and H. R. Lijnen, J Thromb Haemost, 2009; 7(1):4-13). In a further embodiment, varying plasmin concentrations in serum free media were shown to degrade fibrin microfibers at different time points (FIG. 11A). The plasmin concentration ranged from 0.1 to 15 CU/mL. In a preferred embodiment, a 12 hr degradation treatment of 0.25 CU/mL plasmin was able to maintain cell viability similar to control culture conditions while a 24 hr treatment of 0.1 CU/mL plasmin was more detrimental to cell survival possibly due to the longer period of serum starvation (FIG. 11B). Degradation of ECFC-seeded fibrin microfibers after 5 days of culture revealed that both the 12 and 24 hr treatment protocols were able to degrade the fibrin microfiber core while maintaining the intact ECM deposited by ECFCs, resulting in an early microvascular structure comprised of an endothelial layer and its basal lamina with a clear circular lumen (FIG. 11C).

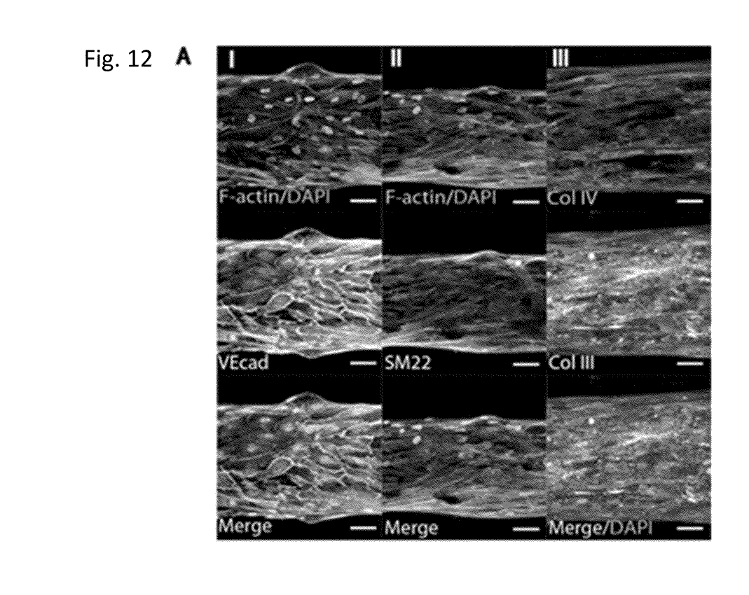

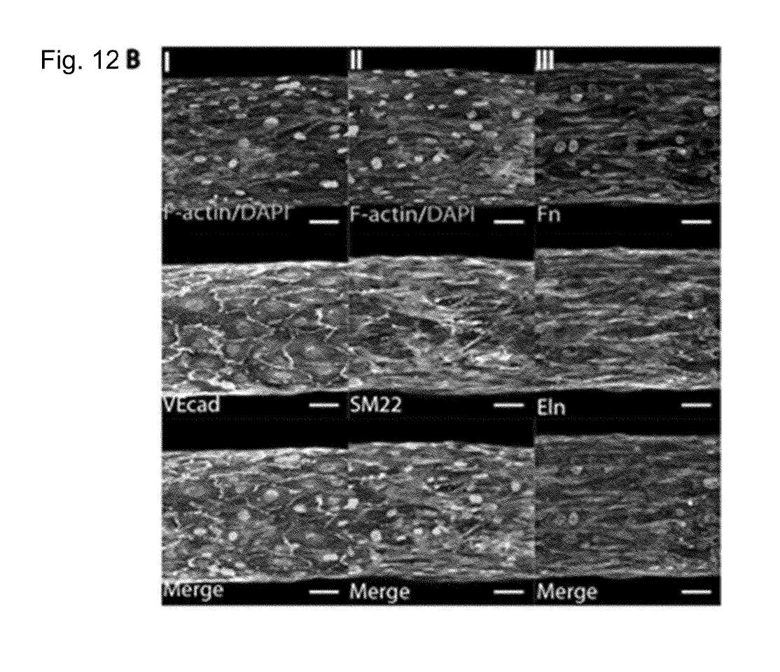

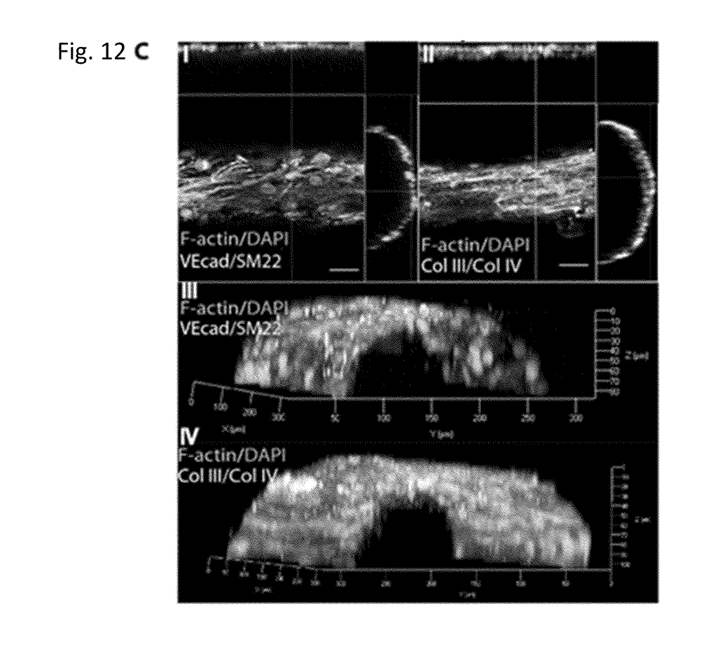

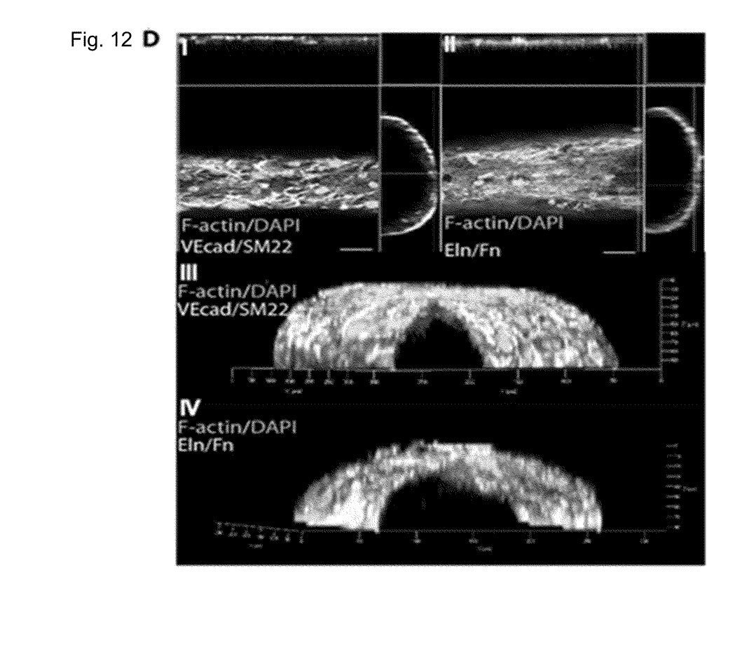

In further embodiment of the invention, luminal multicellular microvascular structures were created by adding perivascular (mural) cells to constructs that first had been cultured with ECFCs to allow full endothelial layer formation before introducing the perivascular (mural) cells. Co-cultures were further cultured and shown to be comprised of both ECFCs and mural cells along with their deposited ECM, such as Col III and Col IV for ECFC-pericyte co-cultures (FIG. 12 a). Unexpectedly, ECFC-vSMC co-cultures presented Eln deposition after only 5 days of vSMC growth (FIG. 12B), an elusive achievement in vascular tissue engineering typically shown in vSMC cultures of over four weeks only (Kim, B.S., et al., Biotechnol Bioeng, 1998, 57(1):46-54; Gao, J., et al., J Biomedical Materials Research Part A, 2008; 85A(4):1120-1128; Patel, A., et al., Cardiovascular Research, 2006; 71(1):40-49; Long, J. L. and R. T. Tranquillo, Matrix Biol, 2003; 22(4):339-50). Finally, the resulting structures were shown to retain both cellular and ECM formation after degradation of the fibrin core, forming a distinct circular lumen (FIGS. 12C, D).

The novel in vitro model system established in accordance with the invention demonstrates the role of vessel diameter on ECM organization during microvascular growth. The system supports a step-wise vascular formation process via introduction of perivascular cells at varying time points, a current challenge in microvascular tissue engineering. This approach can be used to develop further a mechanistic understanding of human microvasculature assembly and stabilization in health and disease.

### ABBREVIATIONS

The following abbreviations may appear in the examples and elsewhere in the specification and claims:

Actin, beta (ACTB)
aminocaproic acid (ACA)
collagen type I (Col I)
collagen, type I, alpha 1 (COL1A1)
collagen type III (Col III)
collagen, type III, alpha 1 (COL3A1)
collagen type IV (Col IV)
collagen, type IV, alpha 1 (COL4A1)
elastin (Eln)
endothelial cells (ECs)
endothelial colony forming cells (ECFCs)
extracellular matrix (ECM)
fibronectin (Fn)
Glyceraldehyde 3-phosphate dehydrogenase (GAPDH)
laminin (Lmn)
Laminin subunit gamma-1 (LAMC1)
polyethersulfone (PES)
smooth muscle cells (SMCs)
three-dimensional (3D)
two-dimensional (2D)
vascular endothelial growth factor (VEGF)
vascular smooth muscles cells (vSMCs)

### EXAMPLES

The invention can be further understood in view of the following non-limiting examples.

Cell Culture

Unless indicated otherwise, the following cells and culture conditions were used in the experiments. Human ECFCs (Lonza, Walkersville, Md.) were used for experiments between passages 5 and 9. ECFCs were expanded in flasks coated with type I collagen (BD Biosciences, Franklin Lakes, N.J.) in Endothelial Basal Medium-2 (EBM-2; Lonza) supplemented with EGM-2 Bulletkit (Lonza) and 10% fetal bovine serum (FBS; Hyclone, Logan, Utah). ECFCs were fed every other day, passaged every 5 to 7 days with 0.05% trypsin/0.1% ethylenediaminetetraacetic acid (EDTA; Invitrogen, Carlsbad, Calif.). Human vSMCs (ATCC, Manassas, Va.) were used between passages 4 and 9 and cultured in F-12K medium (ATCC) supplemented with 0.01 mg/ml insulin (Akron Biotech, Boca Raton, Fla.), 10% FBS (Hyclone), 0.05 mg/ml ascorbic acid, 0.01 mg/ml transferrin, 10 ng/ml sodium selenite, 0.03 mg/ml endothelial cell growth supplement, 10 mM HEPES, and 10 mM TES (all from Sigma-Aldrich, St. Louis, Mo.). Human placental pericytes (Promocell, Heidelberg, Germany) were cultured in Pericyte Growth Media (Promocell) and used between passages 7 and 10. Media was changed every other day and cells were passaged every 5 to 7 days with 0.05% trypsin.

Immunofluorescence Staining and Confocal Microscopy Imaging

Cell-microfiber constructs were fixed with 3.7% formaldehyde (Fisher Chemical, Fairlawn, N.J.) for 15 min, permeabilized with 0.1% Triton X-100 solution (Sigma-Aldrich) in 3.7% formaldehyde for 10 min, washed three times with PBS, and incubated for 1 h at room temperature with the indicated primary antibodies (Table 1). After rinsing with PBS three times, samples were then incubated with the appropriate secondary antibodies or conjugated phalloidin (Table 1) at room temperature for 1 h. Samples were then rinsed with PBS three times, and counterstained with DAPI for 10 min. Z-stack and cross-sectional images were obtained and processed using confocal microscopy (LSM 510 Meta, Carl Zeiss Inc., Thornwood, N.Y.). Epifluorescence images were obtained using an Olympus® BX60 microscope.

TABLE 1
Antibodies Used in the Studies

| Reagent Type | Name | Host | Vendor | Dilution |
|---|---|---|---|---|
| Microtubule | Nocodazol | NA | Sigma-Aldrich | 3.3 μM |
| disrupting | | | | |
| agent | | | | |
| Actin | Cytochalasin D | NA | Sigma-Aldrich | 1 ug/mL |
| disrupting | | | | |
| agent | | | | |
| Primary | CD31 | mouse | Dako | 1:100 |
| Antibody | vWF | mouse | Dako | 1:100 |
| | VEcad | mouse | Santa Cruz | 1:100 |
| | | | Biotechnology | |
| | SM22 | rabbit | Abcam | 1:200 |
| | Collagen I | mouse | Abcam | 1:100 |
| | Collagen III | mouse | Santa Cruz | 1:100 |
| | | | Biotechnology | |
| | Collagen IV | rabbit | Santa Cruz | 1:100 |
| | | | Biotechnology | |
| | Elastin | mouse | Abcam | 1:100 |
| | Fibronectin | rabbit | Sigma-Aldrich | 1:100 |
| | Laminin | rabbit | Abcam | 1:100 |
| Conjugated | DAPI | NA | Roche | 1:1000 |
| Antibody | | | Diagnostics | |
| | Alexa Fluor 488 | shroom | Invitrogen | 1:50 |
| | Phalloidin | | | |
| Secondary | Alexa Fluor 488 | goat | Invitrogen | 1:500 |
| antibody | anti rabbit IgG | | | |
| | Alexa Fluor 546 | donkey | Invitrogen | 1:500 |
| | anti mouse IgG | | | |
| | Alexa Fluor 546 | donkey | Invitrogen | 1:500 |
| | anti rabbit IgG | | | |
| | Alexa Fluor 647 | donkey | Invitrogen | 1:500 |
| | anti rabbit IgG | | | |

Transmission Electron Microscopy

Samples were prepared for transmission electron microscopy (TEM) analysis as described previously (Hanjaya-Putra et al., Blood, 2011; 118: 804-815). Briefly, samples were fixed with 3.7% formaldehyde, 1.5% glutaraldehyde in 0.1 M sodium cacodylate, 5 mM CaCl2, and 2.5% sucrose at room temperature for 1 h and washed 3 times in 0.1 M cacodylate/2.5% sucrose (pH 7.4) for 15 min each. The cells were post-fixed with Palade's 0504 on ice for 1 h, en bloc stained with Kellenberger uranyl acetate overnight, dehydrated through a graded series of ethanol, and then embedded in EPON™ epoxy resin. Sections of 80 nm were cut, mounted onto copper grids, post-stained in 2% uranyl acetate and Reynolds lead citrate, and viewed using a Phillips® EM 420 transmission electron microscope (FEI). Images were captured with an Olympus® Soft Imaging Systems Megaview III CCD digital camera.

Scanning Electron Microscopy

Hydrogel microfiber samples were first serially dehydrated in 50%, 60%, 70%, 80%, 90%, 95% and 100% ethanol for 15 min in each solution, critical point dried, and then sputter-coated with 8 nm thick Au/Pd (gold/palladium particles). Samples were imaged on a field-emission scanning electron microscopy (SEM) (JEOL 6700F, Tokyo, Japan).

Image and Statistical Analyses

The cell areas and perimeters were measured by fitting an ellipse to each cell using the LSM 510 software. Cytoskeletal alignment angle was calculated by measuring the angle between the long axis of each ellipse and the longitudinal axis of the microfiber, found by drawing a line at the edges of the microfiber in its image projection. ECM angle of orientation was measured using the LSM 510 software by drawing a line following the ECM deposition and finding the angle between the line and the longitudinal axis of the microfiber. Graphs were plotted with 5-95% confidence intervals. Unpaired two-tailed Welch-corrected t-tests were performed where appropriate (GraphPad Prism® 5.01, GraphPad Software, San Diego, Calif.). Significance levels were determined between samples examined and were set at *p<0.05, **p<0.01, and ***p<0.001.

### Example 1

### ECFC Attachment and Alignment on Fibrin Microfibers

A new approach to create aligned hydrogel microfibers using an electrostretching process of polymer materials is disclosed. Unique characteristics of the electrostretched hydrogel microfibers are the internal and topographical alignment of the fibrous structure, generated as a result of both electrical field and mechanical shear-induced polymer chain alignment as described above. Furthermore, the diameter of a microfiber is controlled and uniform as a result of the bundling and processing of the individual nanofibers that compose the hydrogel microfibers. Fibrin gels have been extensively used to study microvasculature assembly (Dickinson L E, et al., Soft Matter, 2010; 6: 5109-5119; Bayless, K J, and Davis, G E, Biochemical and Biophysical Research Communications, 2003; 312: 903-913; Davis G E, and Bayless K J, Microcirculation, 2003; 10: 27-44; Bayless K J, et al., RGD-Dependent American Journal of Pathology, 2000; 156: 1673-1683; Dickinson L E, et al., Lab Chip, 2012; 12: 4244-4248), vSMC responses (Ahmann K A, et al., Tissue Eng Part A, 2010; 16: 3261-3270; Long J L, and Tranquillo R T, Matrix Biol, 2003; 22: 339-350) and multicellular organization (Lesman A, et al., Biomaterials, 2011; 32: 7856-7869). Fibrin is used as the matrix material to prepare hydrogel microfibers as a template for the step-wise creation of microvasculature of the invention.

Preparation of 3D Fibrin Hydrogel Microfiber

Fibrin hydrogel microfibers were generated by the electrostretching method (FIG. 1A). An aqueous solution of 1.5 wt % alginate (Sigma-Aldrich) was in-line mixed with 2 wt % fibrinogen (Sigma-Aldrich) at feeding rates (flow rates) of 2 ml/h and 1 ml/h, respectively. Both solutions were dissolved in 0.2 wt % poly(ethylene oxide) (PEO) (Mw=4,000,000, Sigma-Aldrich) prior to spinning. The mixed solution was then charged with 4 kV electric potential and extruded through a 25-gauge needle. The fibrinogen-alginate solution jet was collected, at a distance of about 3-5 cm from the needle tip, in a grounded, rotating bath (20 cm diameter, 20-40 rotation/min) containing 50 mM CaCl2 solution with 5 units/ml thrombin (Sigma-Aldrich) as a cross-linking solution (FIG. 1). After spinning, fibers were left in the collection solution for 15 min. To generate microfibers with different diameters, collection times were varied from 7-80 min, including 7, 10, 15, 17, 20, 22, 26, 27, 35, 40, 45, 50, 55, 60, 70, 75, and 80 min. The crosslinked fibrin-alginate fibers were then soaked in 0.2 M sodium citrate overnight to remove calcium ions and dissolve alginate. Fibers were soaked in water for 30 min to remove sodium citrate, stretched manually to about 150% of their initial length by placing the ends of the microfiber on supports such as pipettes and extending the supports to for example 150% of their original distance, and air-dried for 30 min. Fibers were wrapped around a custom-made plastic frame, then sterilized by soaking in 75% ethanol for 2 min followed by rinsing twice with sterile water. Microfiber diameter was measured from confocal Z-stack projections.

Cell Seeding and Culture on Fibrin Hydrogel Microfibers

ECFCs were seeded on microfibers of 15-cm length total at a density of 4×10⁵ cells/ml in 5 ml of ECFC media supplemented with 50 ng/mL of VEGF (Pierce, Rockford, Ill., USA). The seeding tube was continuously rotated on a tumbler (Labquake, Dubuque, Iowa) for 24 h at 37° C. to facilitate cell attachment. Frames with ECFC seeded microfibers were then transferred to 35 mm Petri dishes using tweezers, and cultured in the same media in a CO2 incubator at 37° C. Media was refreshed every other day thereafter.

Results

Using the electrostretching method, fibrin hydrogel microfibers exhibited longitudinally-aligned nanotopography (FIG. 1A). This is an important feature as sub-micron (<1 μm, but greater than 100 nm) scale topographic features have been shown to increase EC adhesion, migration and orientation (Ranjan A, and Webster T, Nanotechnology, 2009; 20: 305102; Liliensiek S, et al., Biomaterials, 2010; 31: 5418-5426; Bettinger C J, et al., Adv Mater, 2008; 20: 99-103; Lu J, et al., Acta Biomater, 2008; 4: 192-201).

The seeding of ECFCs was facilitated by continuous rotation (FIG. 1A). After 24 hrs, ECFCs attached to the microfibers throughout the surface (FIG. 1E). Within 5 days in culture, ECFCs were found to be elongated and aligned longitudinally with the microfibers as indicated by F-actin staining (FIG. 1B-D). ECFCs covered the microfiber surface continuously, and exhibited typical membrane expression of endothelial markers VEcad and CD31 (FIG. 1B, 1D), and cytoplasmic expression of von Willebrand factor (vWF) (FIG. 1C), demonstrating that fibrin microfibers support the adhesion and culture of ECFCs.

### Example 2

### ECM Deposition from ECFCs on Fibrin Microfibers

While the importance of ECM deposition in vascular development has been recognized, few studies have looked at ECM production by the endothelium. ECFCs deposit collagen IV, fibronectin and laminin in an organized web-like structure when cultured on Petri dishes (Kusuma S, et al., FASEB J, 2012; 26: 4925-4936). To establish a reliable in vitro model of microvasculature, we characterized the ECM protein deposition by the endothelium on hydrogel microfibers.

ECFCs were seeded on fibrin hydrogel microfibers as described in Example 1.

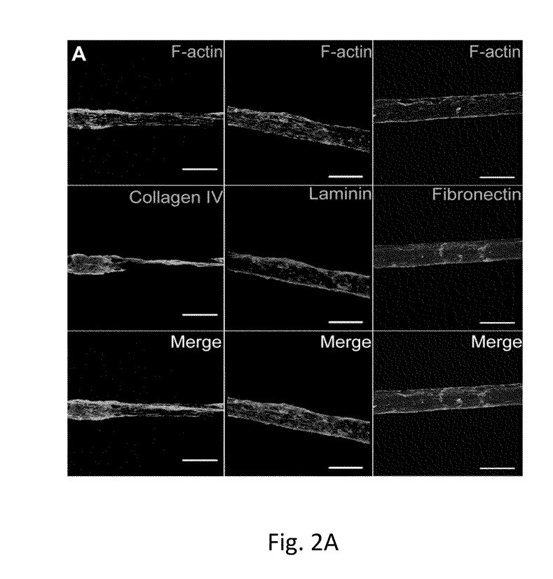

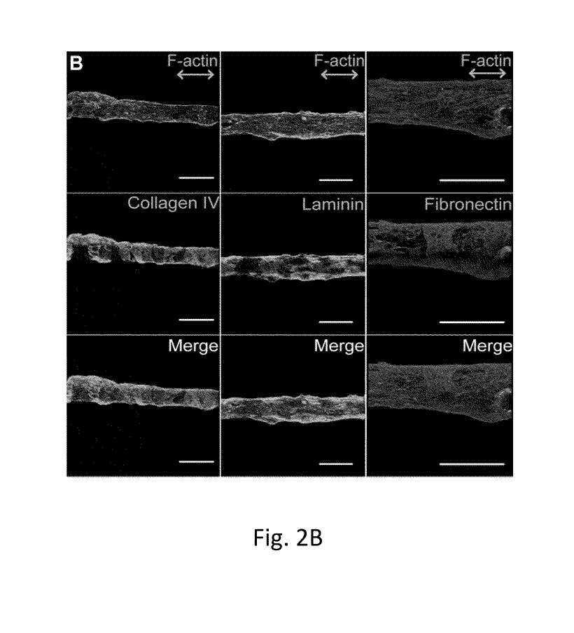

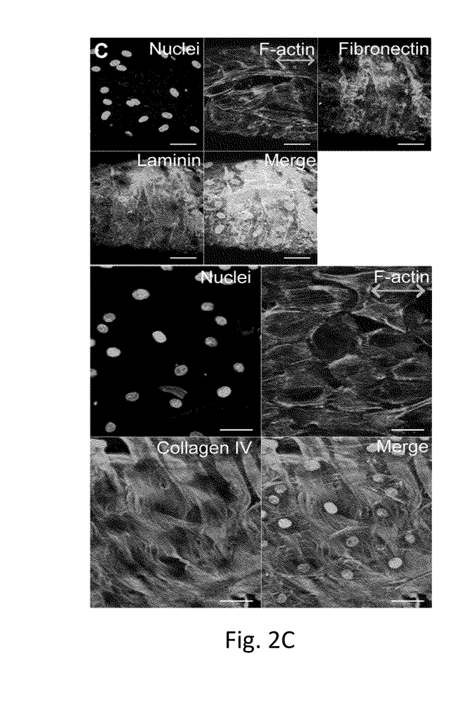

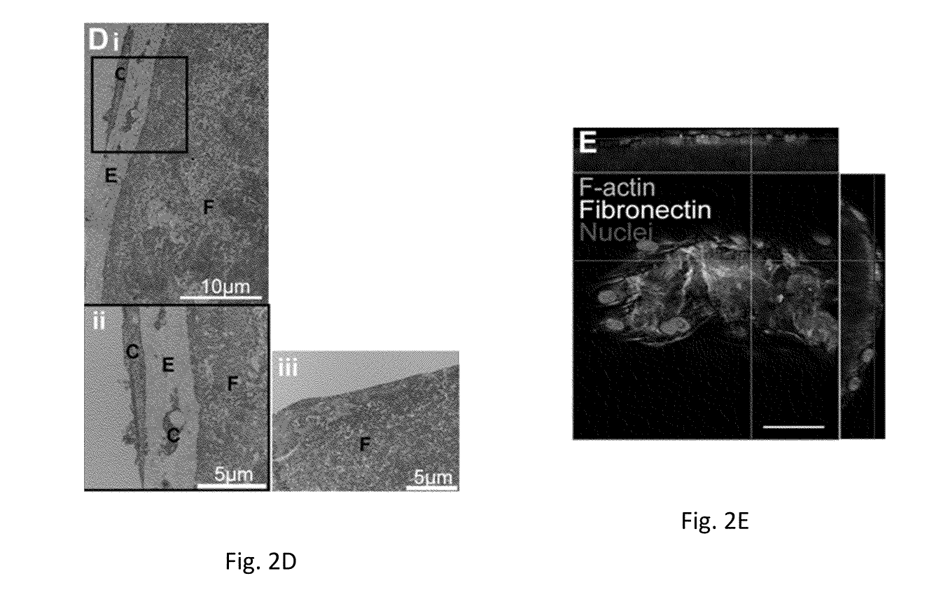

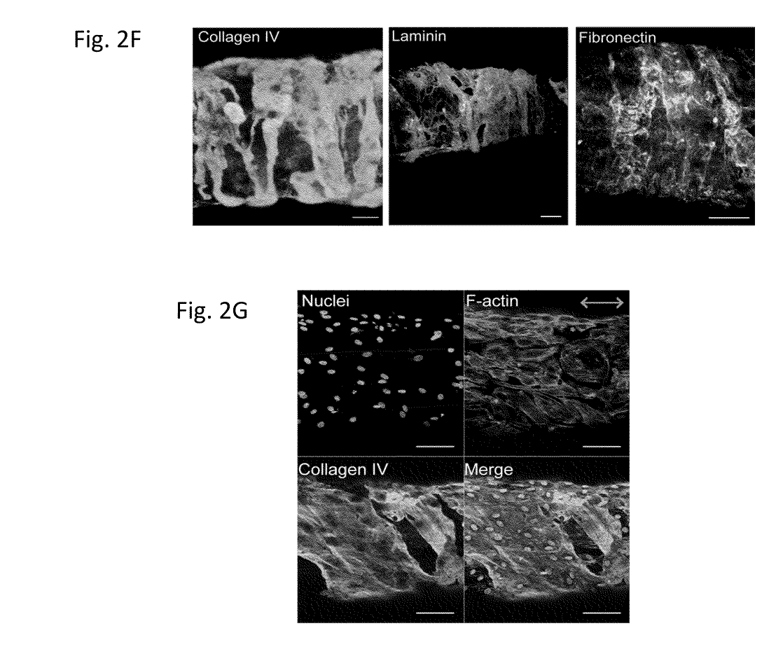

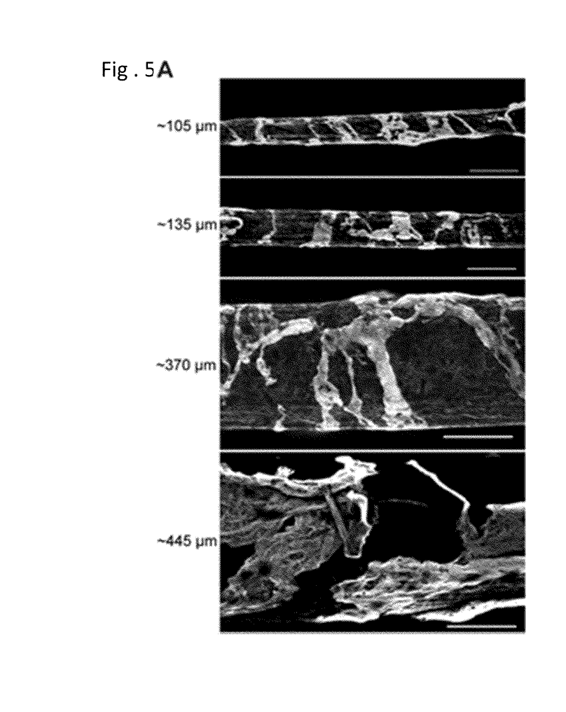

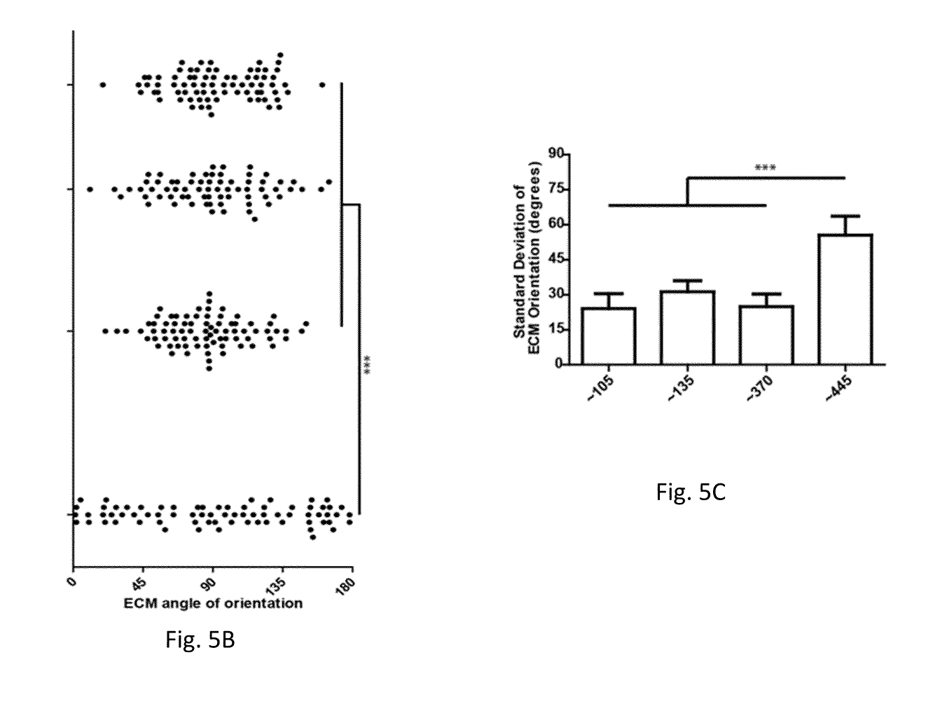

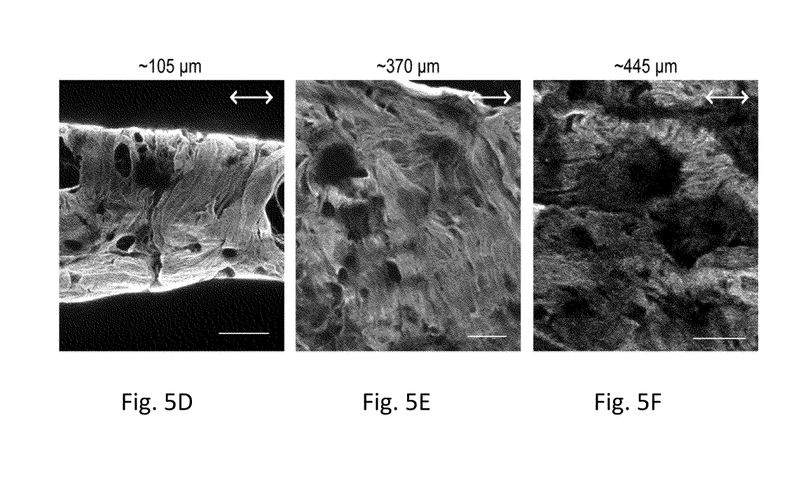

ECFC seeded on fibrin microfibers deposited laminin, collagen IV, and fibronectin after one day in culture (FIG. 2A). On Day 5, ECFCs completely covered the fibrin microfiber, and abundant ECM deposition was observed (FIG. 2B). In contrast to what was observed previously on Petri-dishes (Kusuma S, et al., FASEB J, 2012; 26: 4925-4936), the ECM proteins deposited by ECFCs on hydrogel microfibers were organized. ECM proteins laminin, collagen IV, and fibronectin wrapped around the microfibers, perpendicular to the EC orientation, along the microfiber's circumference, as observed in (FIGS. 2C, 2F, and 2G). This is in contrast to the web-like structure observed on Petri-dishes. Specifically, on fibrin microfibers, the individual collagen IV nano-fibrils also seem to follow this macroscopic circumferential alignment at the nano-scale, with fibrils being deposited next to each other in an aligned manner to create a ribbon of circumferentially aligned collagen (FIG. 2C, 2G). Further analysis revealed this was also true for microfibers of sizes up to about 370 μm, and that the largest microfibers tested (about 445 μm) did not have a distinguishable collagen nano-fibril orientation (FIG. 5D-F). On Petri-dishes, ECM nano-fibrils were deposited without any apparent alignment, in a web-like structure formation (Kusuma S, et al., FASEB J, 2012; 26: 4925-4936). Moreover, these ECM structures appeared to be distributed either below or among the ECFCs (FIG. 2D-E), resembling basal lamina organization found in native microvessels. It should be noted that similarly to what we observed on Petri-dishes (Kusuma S, et al., FASEB J, 2012; 26: 4925-4936), ECFCs did not express or deposit Collagen I (data not shown).

### Example 3

### ECM Deposition from ECFCs on Fibrin Sheets and PES Fibers

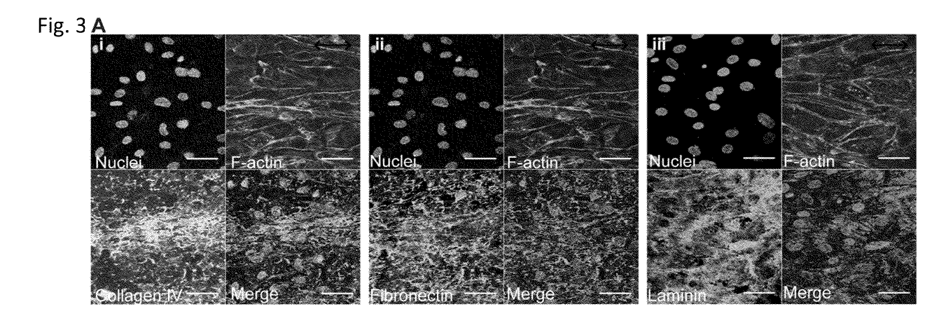

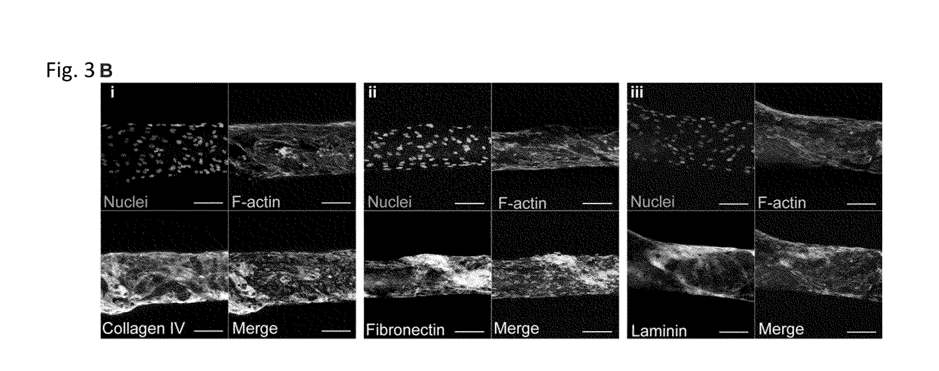

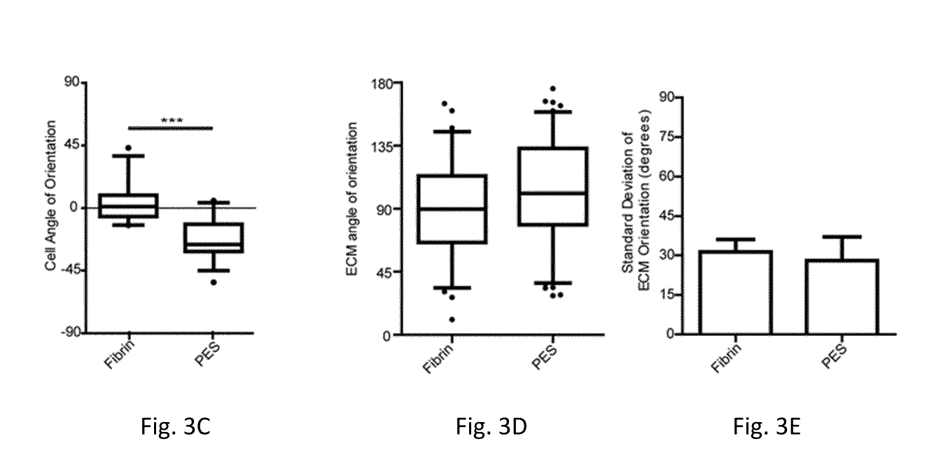

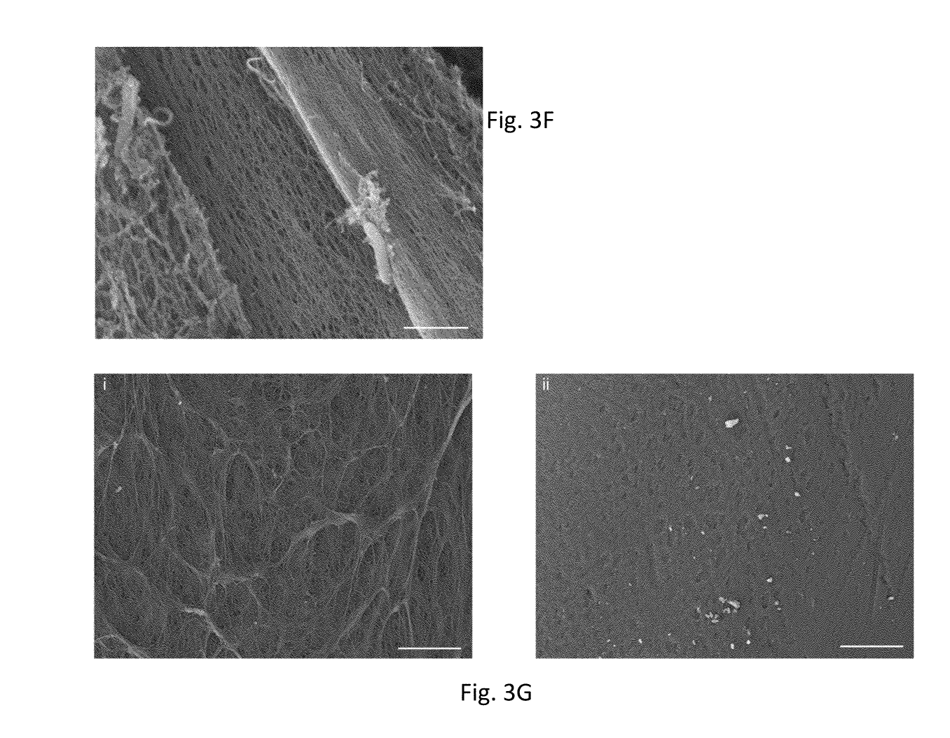

It was previously demonstrated that line-grating topography influences EC adhesion, alignment, and elongation (Ranjan A, and Webster T, Nanotechnology, 2009; 20: 305102; Liliensiek S, et al., Biomaterials, 2010; 31: 5418-5426; Bettinger C J, et al., Adv Mater, 2008; 20: 99-103; Lu J, et al., Acta Biomater, 2008; 4: 192-201). To probe if the aligned nanotopography on fibrin microfibers is responsible for ECFC alignment and coordinated deposition of laminin, collagen IV, and fibronectin, we first examined their deposition on flat (2D) fibrin sheets with similar aligned nanotopography (FIG. 3E) as the fibrin hydrogel microfibers by varying the dimensionality and cylindrical shape of the scaffold.

Preparation of 2D Fibrin Nanofiber Sheets

Fibrin-alginate hydrogel nanofibers were prepared according to the same electrostretching method as described in Example 1 above until the collection step in a rotating bath. The collected fibrin-alginate hydrogel nanofibers were then wrapped around a modified plastic frame to form a sheet of hydrogel nanofibers while slightly stretching the nanofibers to ensure proper alignment. The fibrin nanofiber sheets were placed in a 0.2 M sodium citrate solution overnight to remove alginate, followed by a 30 min wash in water to remove excess sodium citrate. Fibrin nanofiber sheets were then sterilized with 75% ethanol and rinsed twice with sterile water. The resulting nanofiber sheets are distinct from microfibers as these are not bundled, stretched, and air-dried to form a microfiber, but instead are collected from the rotating bath in a 2D nanofiber sheet formation.

Cell Seeding and Culture on 2D Fibrin Sheets

Cells were seeded by placing 5×10⁵ ECFCs in a concentrated solution of cells (about 2×10⁶ cells/ml) directly on top of the fibrin nanofiber sheet. After 5 min the cell solution that had filtered through the sheets was collected and reseeded on top of the fibrin nanofiber sheets. This process was repeated 3 times, after which the same culture media as used for 3D fibers was added to the samples before they were placed in a humidified incubator at 37° C. in a 5% CO2 atmosphere. Media was refreshed every other day up to 5 days of culture.

Preparation of 3D Polyethersulfone Fibers

Solid polymer fibers were prepared as a control according to a modified electrospinning protocol. In brief, polyethersulfone (PES) (Goodfellow Cambridge Limited, UK, Mw 55,000) was dissolved in 30 wt % DMSO and electrospun under an electric potential of 5 kV. The feed rate of PES solution was 12 ml/h to initiate a polymer jet, which was collected in a grounded, rotating ethanol bath (20-40 rotations/min) to extract the solvent. The collection distance was set to 5 cm. After 10 min in ethanol, PES strings were removed from the bath and air-dried.

After electrospinning, PES fibers were wrapped around a seeding frame similarly to the fibrin hydrogel microfibers. Samples were then plasma-treated for 5 min before soaking for 5 min in a 10 units/ml thrombin in 15 mM CaCl2 solution. Thrombin-coated PES fibers were then immersed in a 0.2% fibrinogen solution diluted in 0.9% NaCl for fibrinogen polymerization into fibrin. Excess fibrin coating on the frame and outside of the fibers was removed before sterilization with 75% ethanol for 1-2 min. Samples were rinsed twice with sterile water, after which cell seeding was performed similarly to the fibrin microfibers as described in Example 1.

Results

ECFCs seeded on fibrin nanofiber sheets were effectively aligned with the nanotopography. However, the collagen IV, fibronectin and laminin produced by ECFCs exhibited a random organization, as opposed to the perpendicular orientation with respect to cell alignment observed in the 3D microfibers (FIG. 3A). This result indicates that the microfiber geometry may be crucial to the specific organization of ECM molecules secreted by ECFCs.

Similarly as observed in FIG. 2, ECFCs completely covered the PES microfibers and deposited ECM molecules after 5 days of culture (FIG. 3B). While ECFCs grown on the PES microfibers did not necessarily have a random orientation on the PES fibers, and in fact often exhibited a partial diagonal orientation (FIG. 3C), the ECFC-deposited ECM proteins were found to wrap around the PES microfiber in a similar manner to ECFC-deposited ECM on fibrin microfibers, as evidenced by measuring the angles between ECM ribbons and the fiber's longitudinal axis (FIG. 3D). Note that the average angle in both cases is close to 90 degrees, demonstrating perpendicular alignment, and that the distribution (represented by the height of the boxes and also shown as standard deviation in (FIG. 3E) is small, signifying most ECM ribbons had an orientation close to 90 degrees.

Electrospun PES microfibers coated with fibrin used to generate microfibers maintain the dimensionality and geometry of the fibrin microfibers but with a random nanotopography (FIG. 3F). Before coating the PES fibers with fibrin, the surface of PES fibers is smooth (FIG. 3G (ii)), however, an uncoated PES fiber is not bioadhesive, and ECFC attachment after seeding is not detected (data not shown). Furthermore, PES fiber does not present the same bioactive substrate to the ECFCs as the fibrin fibers. Therefore, the PES fibers were coated with fibrin, resulting in the random, non-aligned nano-topography of coating presented in FIG. 3G (i). Such PES microfibers have a similar diameter (240±45 μm; data not shown) as fibrin microfibers and thus enables investigation of whether the uniaxial alignment topography contributes to the unique cellular activity and ECM organization.

### Example 4

### ECM Organization: Dependence on ECFC Alignment and Microtubule Organization

Actin and microtubule disruption studies were used to determine whether ECFC actin filament alignment and microtubule organization through actin and tubulin configuration directs ECM organization. Cytochalasin D is an actin destabilizing agent, and nocodazole is a microtubule polymerization disturbing agent.

Cell Seeding and Culture

ECFCs were seeded on fibrin microfibers as described in Example 1 above.

Actin and Microtubule Disruption Studies

Cytochalasin D or nocodazole were dissolved in DMSO (Table 1). ECFCs were cultured on Petri dishes (control) or fibrin microfibers in ECFC media with 50 ng/mL of VEGF (Pierce, Rockford, Ill., USA) supplemented with either 1 g/mL cytochalasin D or 3.3 M nocodazole, from either day 0 or day 1 after seeding. F-actin or a-tubulin organization and ECM deposition was analyzed after 1, 2, or 3 days of treatment. Final concentration of DMSO in cell culture medium was kept at 0.1% (v/v). Controls were treated with DMSO alone at the same concentration.

Results

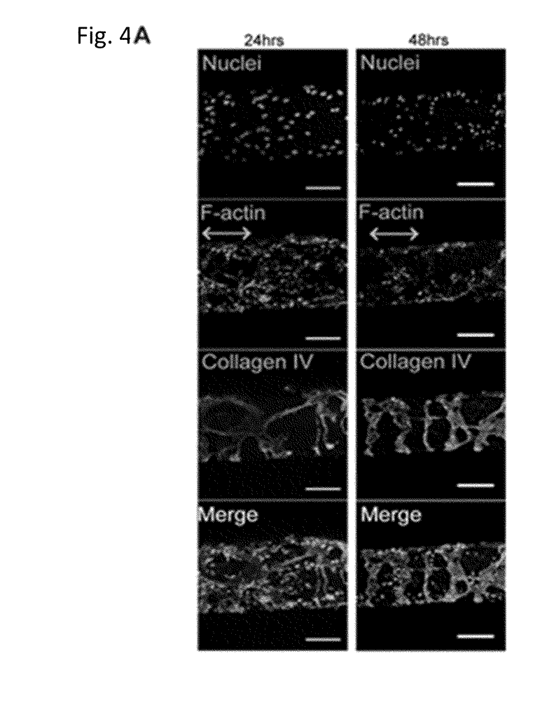

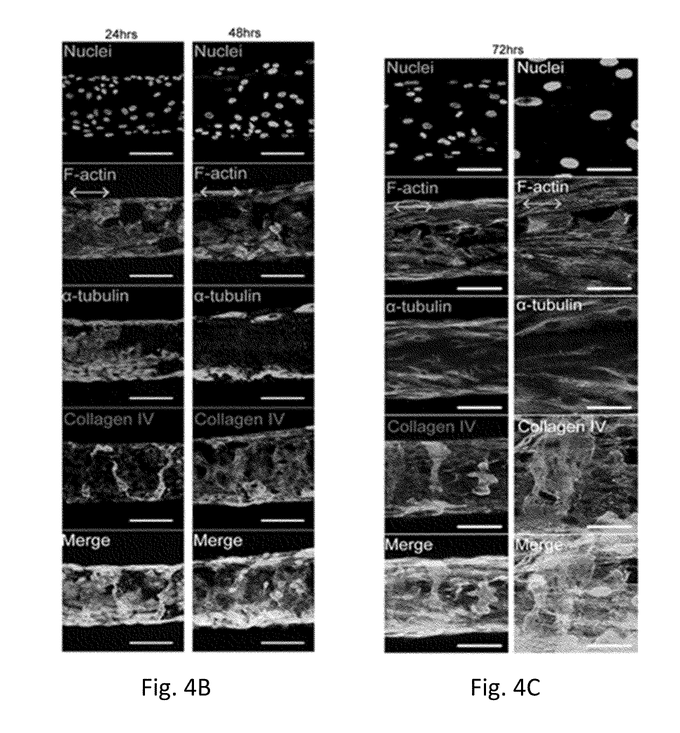

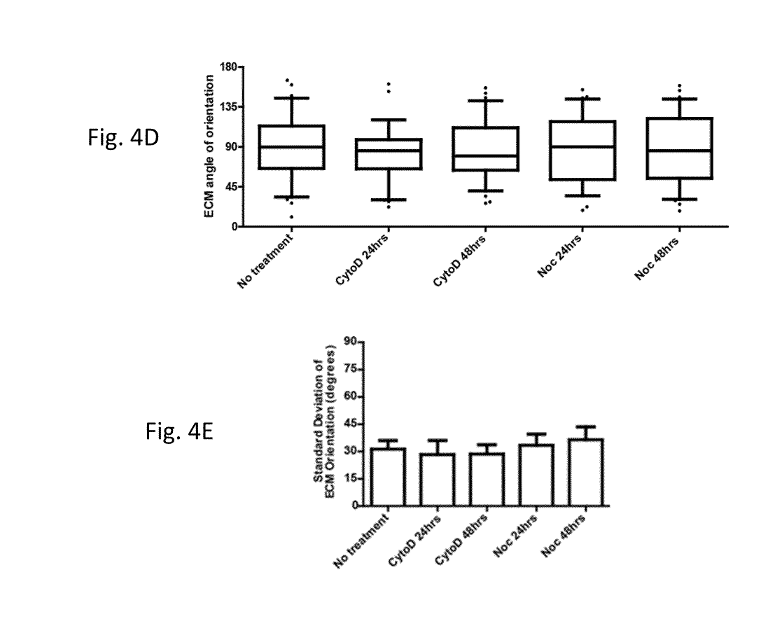

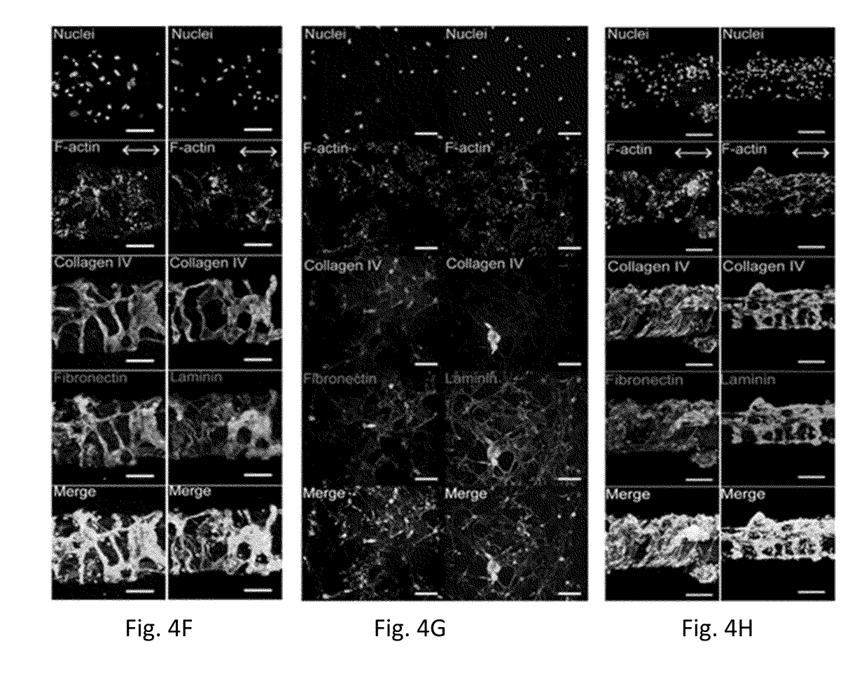

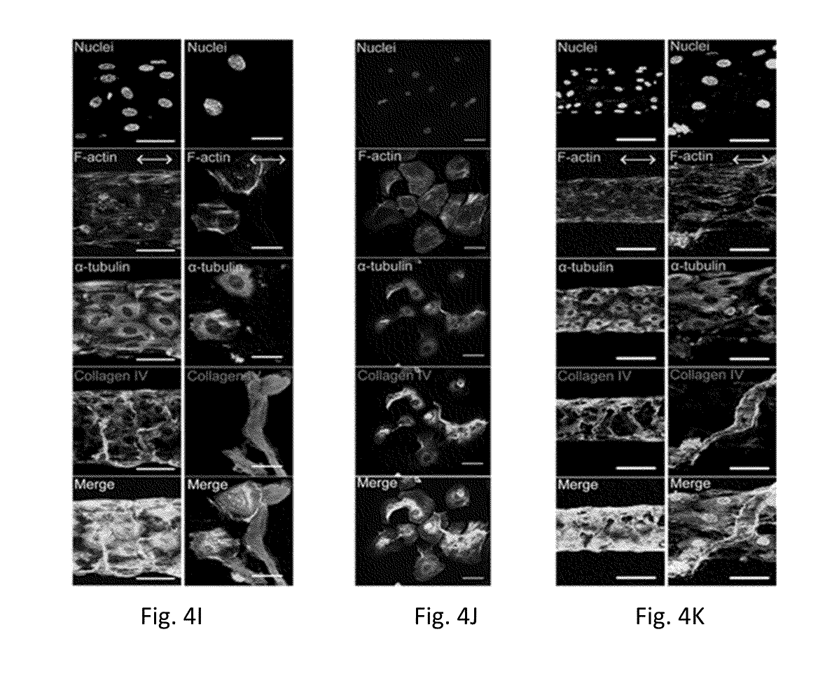

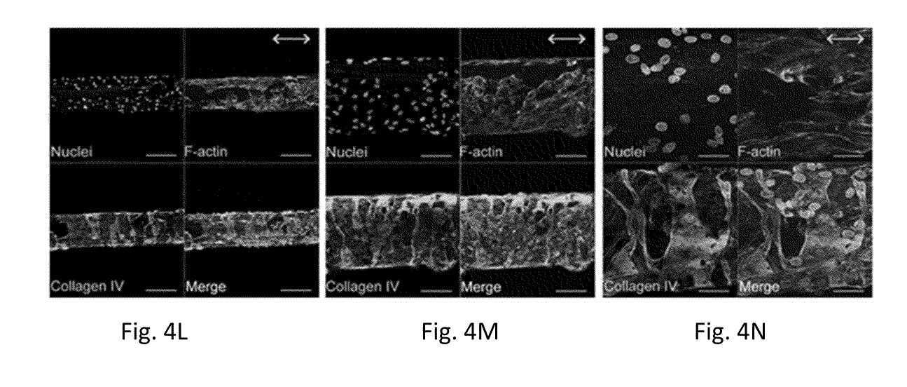

For Cytochalasin D added to the culture media at 24 h after ECFC seeding on microfibers, forty-eight hours after treatment the unique wrapping arrangement of the deposited ECM molecules around the fibrin microfibers still present after 24 and 48 hrs of treatment (FIG. 4A and FIG. 4F). No difference was found in the average angle of ECM orientation or in the variance of all angles (FIG. 4D-E). It should be noted that ECM deposition was observed also in control treatments of ECFCs in Petri dishes (FIG. 4G) and that similar organization of wrapping ECM was observed when cytochalasin D was applied through the entire 3 days of culture (FIG. 4H).

Likewise, when nocodazole, a microtubule polymerization disturbing agent, was added to the culture media at 24 h after ECFC seeding on microfibers, a similar ECM wrapping pattern was observed (FIG. 4B; FIG. 4I) even though microtubule formation was disrupted compared to samples with no drug treatment (FIG. 4C). Similarly, no difference was found in the average angle of ECM orientation or in the variance of all angles measured (FIG. 4D-E). In addition, ECM deposition was also observed in the 2D control group with the same treatment (FIG. 4J), and similar organization of wrapping ECM was observed when Nocodazole D was applied through the entire 3 days of culture (FIG. 4K).

Overall, while treatment of ECFCs seeded on the nanopatterned microfibers with either cytochalasin D or nocodazole effectively altered actin and microtubule organization, respectively, it did not alter the wrapping organization of the deposited ECM molecules as compared to control cultures (FIG. 4). Also, even after only 3 days of culture when the endothelium layer was not always confluent and therefore the cell density was lower, ECM organization was still found to be circumferential (FIG. 4C-E, FIG. 4L-M).

### Example 5

### ECM Organization: Dependence on Microfiber Curvature

As indicated, the ECM organization is independent of the cytoskeleton organization of the ECFCs, but is influenced by the geometry of the microtubular structure. Here the diameter of the tubular structure to modulate the ECM organization was evaluated.

Preparation of Fibrin Hydrogel Microfiber

Hydrogel microfibers were prepared as in Example 1 above. Microfibers of different sizes were prepared by varying the collection time of the electrostretching process, thus changing the number of nanofibers in each microfiber bundle. Fibrin microfibers with an average diameter of 107.1±11.7 μm, 136.1±12.1 μm, 372.0±27.3 μm, and 443.4±30.6 μm were prepared. These microfibers were processed similarly and thus exhibited similar nanotopographical alignment, inducing alignment of ECFCs with the longitudinal axis of the microfibers on all sizes (data not shown).

Results

While seeded-ECFCs were confluent after 5 days in culture on all microfibers, decreasing organization of the ECM molecules was apparent as the fiber diameter increased (FIG. 5A). Measuring the angles between ECM ribbons and the microfiber's longitudinal axis revealed that microfibers with diameters smaller than about 400 μm had average angles close to 90° with a small distribution, signifying perpendicular orientation. On the largest diameter tested (avg. 452.1±26.7 μm), we observed a non-circumferential ECM organization as evidenced by a significant increase in the distribution of the angles between ECM ribbons and the fiber's longitudinal axis (FIG. 5B). Since the angle values measured range from 0° to 180°, a perfectly random distribution of angles would average to the mean of 90° as well. However, a sample with ECM wrapping would have most of these values close to 90°, having a small variance compared to a sample presenting random ECM orientation. Indeed, the largest microfibers resulted in ECM angles with a markedly higher standard deviation compared to the smaller microfibers (FIG. 5C).

Also, when collagen IV has a wrapping organization, the individual collagen nano-fibrils also follow this macroscopic circumferential alignment at the nano-scale (FIG. 2C, 2G). This was also true for microfibers of about 105 μm to about 370 μm, and the largest microfibers tested (about 445 μm) did not have a distinguishable collagen nano-fibril orientation (FIG. 5D-F).

### Example 6

### vSMCs and Pericyte Attachment and New ECM Deposition on ECFC-Seeded Microfibers

An advantage of the new fibrin microfiber system is the opportunity to co-culture mural cells, for example, vSMCs and pericytes, to study their interactions with the endothelial layer as well as the deposition of ECM component that compose the tunica media (Jain R K, Nat Med, 2003; 9: 685-693; Carmeliet P, Nature, 2000; 407: 249-257). Here, ECFC-seeded fibrin microfibers are co-cultured with vSMCs and/or pericytes to evaluate the effect on organization and ECM deposition.

Cell Seeding and Culture

ECFC-seeded fibrin hydrogel microfibers were prepared as described in Example 1 above.

Human vSMCs (ATCC, Manassas, Va.) were used between passages 4 and 9 and cultured in F-12K medium (ATCC) supplemented with 0.01 mg/ml insulin (Akron Biotech, Boca Raton, Fla.), 10% FBS (Hyclone), 0.05 mg/ml ascorbic acid, 0.01 mg/ml transferrin, 10 ng/ml sodium selenite, 0.03 mg/ml endothelial cell growth supplement, 10 mM HEPES buffer, and 10 mM TES buffer (all from Sigma-Aldrich, St. Louis, Mo.).

vSMCs were seeded on 5-7 day ECFC-seeded fibrin microfibers at 1−4×10⁵ cells/ml in 5 ml of 0.5% serum or regular ECFC media, tumbled for 24 hours, and then transferred to 35 mm Petri dishes to continue culture. Media was changed every other day thereafter.

Human pericytes (PromoCell, Heidelberg, Germany) were used between passages 5 and 9 and cultured in Pericyte Growth Media (PromoCell) according to manufacturer's instructions. Pericytes were seeded on 5 day ECFC-seeded fibrin microfibers at 4×10⁵ cells/ml in 5 ml of ECFC media supplemented with 30 mM aminocaproic acid (to prevent fibrinolysis), tumbled for 24 hours, and then transferred to 35 mm Petri dishes to continue culture for 9 more days (10 days of co-culture total). Media was changed every other day.

Results

vSMCs seeded on ECFC-coated fibrin microfibers attached and grew on the ECFC layer, forming a bilayer cellular construct (FIG. 6A). Occasionally, vSMCs were found to have a random orientation on the structures (FIG. 6A), and in some instances they wrapped around the microfibers (FIG. 6B). However, more often they aligned with the longitudinal axis of the microfibers (FIG. 6C). vSMCs cultured for 3 and 5 days deposited collagen type I and elastin (FIG. 6D-G), located beneath the vSMC layer and above SM22 negative cells, the ECFCs (FIG. 6E, G).

Pericytes cultured for 10 days on ECFC-seeded fibrin microfibers attached and grew on the microfiber scaffold, forming a multilayer cellular construct (FIG. 7). Pericytes either wrapped or aligned with the longitudinal axis of the microfibers (FIGS. 7A-C). Pericytes deposited collagen type IV, located beneath and in between the pericyte layer, and above the ECFCs (FIGS. 7D-E).

### Example 7

### 3D Multicellular Microvascular Structure

Following the successful culture of different vascular cell types, namely ECFCs, pericytes, and vSMCs on fibrin microfibers, increased ECM protein production by the cells was investigated. Also, the fibrin core of the microfiber was degraded leaving both cellular and ECM formation intact, resulting in multicellular microvascular structures with a distinct circular lumen that recapitulate the multilayer organization found in native blood vessels.

Cell Culture

All cells were cultured in a humidified incubator at 37° C. and 5% CO2. Human ECFCs (Lonza, Walkersville, Md.) were cultured on collagen I (BD Biosciences, Franklin Lakes, N.J.) coated flasks in Endothelial Basal Medium-2 (EBM-2; Lonza) supplemented with EGM-2 Bulletkit (Lonza) and 10% fetal bovine serum (FBS; Hyclone, Logan, Utah) and used for experiments between passages 7 and 10. Media was changed every other day and cells were passaged every 5 to 7 days with 0.05% trypsin (Invitrogen, Carlsbad, Calif.).

Human vSMCs (ATCC, Manassas, Va.) were used between passages 7 and 10 and cultured in F-12K medium (ATCC) supplemented with 0.01 mg/ml insulin (Akron Biotech, Boca Raton, Fla.), 10% FBS (Hyclone), 0.05 mg/ml ascorbic acid, 0.01 mg/ml transferrin, 10 ng/ml sodium selenite, 0.03 mg/ml endothelial cell growth supplement, 10 mM HEPES, and 10 mM TES (all from Sigma-Aldrich, St. Louis, Mo.). Media was changed every third day and cells were passaged every 5 to 7 days with 0.25% trypsin (Invitrogen).

Human placental pericytes (Promocell, Heidelberg, Germany) were cultured in Pericyte Growth Media (Promocell) and used between passages 7 and 10. Media was changed every other day and cells were passaged every 5 to 7 days with 0.05% trypsin.

Preparation of 3D Fibrin Hydrogel Microfiber

Fibrin hydrogel microfibers were generated by the electrostretching method described in Example 1. Briefly, 1.5 wt % alginate (Sigma-Aldrich) was mixed in-line with 2 wt % fibrinogen (Sigma-Aldrich) at flow rates of 2 ml/h and 1 ml/h, respectively. Both solutions were dissolved in 0.2 wt % poly(ethylene oxide) (PEO) (Mw=4,000,000, Sigma-Aldrich) prior to electrospinning A 4 kV electric potential was applied to the solution before extrusion through a 25-gauge needle. The solution jet was collected in a grounded, rotating bath (30-45 rotation/min) containing 50 mM CaCl2 solution with 10 units/ml thrombin (Sigma-Aldrich) for 35 min. Fibers were left in the collecting solution for 10 min and then soaked overnight in 0.25 M sodium citrate to dissolve the alginate. Fibers were then soaked in water for 60 min, bundled and stretched to 150% of their initial length, and air-dried for 60 min. Microfibers were wrapped around a custom-made plastic frame and sterilized by soaking in 75% ethanol for 2 min followed by rinsing twice with sterile water.

Cell Seeding and Culture on Fibrin Hydrogel Fibers

ECFCs, vSMCs, and pericytes were seeded on fibers as previously described in Examples 1 and 6. Briefly, cells were seeded on microfibers wrapped on a frame at a density of 4×10⁵ cells/ml in 5 ml of ECFC media and tumbled overnight at 37° C. to facilitate cell attachment. For ECFCs, media was supplemented with 50 ng/mL of vascular endothelial growth factor (VEGF; Pierce, Rockford, Ill., USA), whereas for pericytes media was supplemented with 30 mM aminocaproic acid (ACA; Sigma-Aldrich). At day 2 frames were transferred to 35 mm Petri dishes and cultured in the same media in a 5% CO2 humidified incubator at 37° C. Media was changed every other day thereafter. For co-cultures, vSMCs or pericytes were seeded on 5 day ECFC-seeded fibrin fibers at 4×10⁵ cells/ml in 5 ml of ECFC media or in ECFC media with 30 mM ACA, tumbled for 24 hours, and then transferred to 35 mm Petri dishes to continue culture. Media was changed every other day thereafter.

Preparation of 2D Fibrin Coated Surfaces

Cell culture 6-well plates and coverslips were coated with a solution of 0.2% and 0.1% fibrinogen in normal saline, respectively, and then crosslinked with 10 U/mL thrombin in 15 mM CaCl2. The solutions were incubated for 15 min before being sterilized with 75% ethanol for 2 min and rinsed twice with water.

Cell Seeding and Culture on 2D Fibrin Surfaces

Cells were seeded at 1×10⁵ on 6-well plates and at 5×10⁴ on coverslips in the same culture media as their 3D counterpart. Samples were placed in a humidified incubator at 37° C. in a 5% CO2 atmosphere and media was refreshed every other day up to 5 days of culture.

Plasmin Treatment

Fibrin microfibers with or without cells were treated with 15, 1, 0.25, and 0.1 CU/mL plasmin from human plasma (Athens Research and Technology, Athens, Ga.) in DMEM (Life Technologies, Grand Island, N.Y.) for 1, 6, 12, and 24 hrs respectively in a humidified incubator at 37° C. in a 5% CO2 atmosphere. Samples were imaged immediately after treatment.

Live/Dead Assay

ECFCs cultured in 2D and treated with plasmin were incubated with 2 μM calcein AM and 4 μM ethidium homodimer (Invitrogen) in PBS for 30 min at 37° C. and 5% CO2. Samples were imaged immediately after and the number of live and dead cells was counted using ImageJ (NIH, Bethesda, Md.).

Immunofluorescence Staining and Imaging

Samples were processed as previously described above.

Reverse Transcription Polymerase Chain Reaction

Two-step RT-PCR was performed on 2D and 3D constructs as previously described (Wanjare, M., S. Kusuma, and S. Gerecht, Stem Cell Reports, 2014; 2(5): 561-575). Briefly, total RNA was extracted using a TRIzol protocol (Gibco, Invitrogen) and quantified using an ultraviolet spectrophotometer. RNA was transcribed at 1 μg per sample using reverse transcriptase MMLV (Promega Co., Madison, Wis.) and oligo(dT) primers (Promega) according to manufacturer's instructions. TaqMan Universal PCR Master Mix and Gene Expression Assay (Applied Biosystems, Foster City, Calif.) were used to quantitate the expression of COL1A1, COL3A1, COL4A1, FN1, ELN, LAMC1, ACTB, GAPDH, and 18S genes (Life Technologies). The PCR step was performed for 40 cycles of 15 seconds at 95° C. and 1 min at 60° C. in an Applied Biosystems StepOne Real-TimePCR System (Applied Biosystems). Relative expressions of the genes were normalized to the amount of ACTB, GAPDH, or 18S in the same cDNA by using the comparative AACT method provided by the manufacturer. Samples were run in triplicate.

Statistical Analyses

All experiments were performed in triplicate for at least 2 biological replicates. Paired t-tests were performed to compare significance between 2D and 3D gene expression (GraphPad Prism 5.01, GraphPad Software, San Diego, Calif.) and graphs were plotted with SEM. Significance levels were determined between samples examined and were set at *p<0.05, **p<0.01, and ***p<0.001.

Results

Deposition of ECM Proteins by ECFCs in 3D vs 2D Substrates

We had originally shown the ability of ECFCs to deposit basal lamina proteins Col IV, Fn, and Lmn when cultured in two-dimensional (2D) petri dishes (Kusuma S, et al., FASEB J, 2012; 26: 4925-4936). We also demonstrated in Example 5 above the importance of micro-scale curvature in regulating the organized deposition of these ECM proteins by ECFCs utilizing fibrin microfibers of different sizes. Specifically, we showed that microfibers with diameters ranging from 100 to 400 nm guide circumferential ECM deposition by ECFCs. Here, the 3D fibrin microfibers (average diameter 188.2±13.9 μm) upregulate the amount of ECM proteins produced by ECFCs. As shown in FIG. 8, ECFCs deposit wrapping Col IV, Fn, and Lmn after 5 days in culture on 3D fibrin microfibers and 2D fibrin-coated substrates. However, higher amounts of all ECM proteins were observed in 3D vs 2D (FIG. 8A-B). Quantitative RT-PCR performed on these substrates also revealed a higher expression of the genes encoding these ECM proteins in 3D than in 2D (FIG. 8C). Together, these results show that the fibrin microfibers promote not only proper ECM organization, but also increase ECM deposition compared to 2D cultures.

Deposition of ECM Proteins by Pericytes in 3D vs 2D Substrates

Regulation of the quantity of ECM proteins deposited by perivascular cells by substrate dimensionality was shown with pericytes seeded on fibrin microfibers following the same protocol used for ECFCs. Notably, pericytes were capable of degrading fibrin microfibers before 5 days, a finding in line with the demonstrated fibrinolytic effects of other mural cells (Grassl, E. D., et al., J. Biomedical Materials Res., 2002. 60(4):607-612) but contradictory to studies that have found pericytes to express fibrinolysis inhibitors (Kim, J. A., et al., Brain endothelial hemostasis regulation by pericytes. J Cereb Blood Flow Metab, 2005. 26(2): 209-217). To allow study of ECM deposition after 5 days in culture the media was supplemented with 30 mM ACA, which has been previously used to inhibit plasmin activity 9 (Ahmann, K. A., et al., Tissue Eng Part A, 2010. 16(10): 3261-70; and Anonick, P. K., et al., Arterioscler Thromb, 1992. 12(6):708-716.

As shown in FIG. 9A, pericytes attach and grow on fibrin microfibers. Similarly to ECFCs, pericytes also produce Col IV, Fn, and Lmn both in 2D and 3D. Additionally, they deposit Col I and Col III (FIG. 9A-B). Eln deposition was not observed neither in 2D nor 3D cultures as confirmed by RT-PCR (FIG. 9C). However, the amount of ECM deposited by pericytes was not distinguishably different in 2D vs 3D based on confocal microscopy. Quantitative RT-PCR analysis revealed there is an increased expression of COL1A1,COL3A1, COL4A1, FN1 and ELN, but that this increase is variable and not statistically significant (FIG. 9C).

ECM deposition in 2D appears either randomly organized (FIGS. 9B I and III), non-polymerized (FIGS. 9B II and V), or following local pericyte orientation (FIG. 9B IV), whereas in 3D ECM deposition is organized parallel to the cell's orientation, which follows the longitudinal axis of the microfiber (FIG. 9A). Additionally, Lmn shows a polymerized extracellular deposition in 3D compared to 2D (FIGS. 9A V, 9B V, and 9D). Lastly, pericytes can grow in a multilayer organization on the microfibers (FIG. 9D), as opposed to the monolayer formed by ECFCs.

Deposition of ECM Proteins by vSMCs in 3D vs 2D Substrates

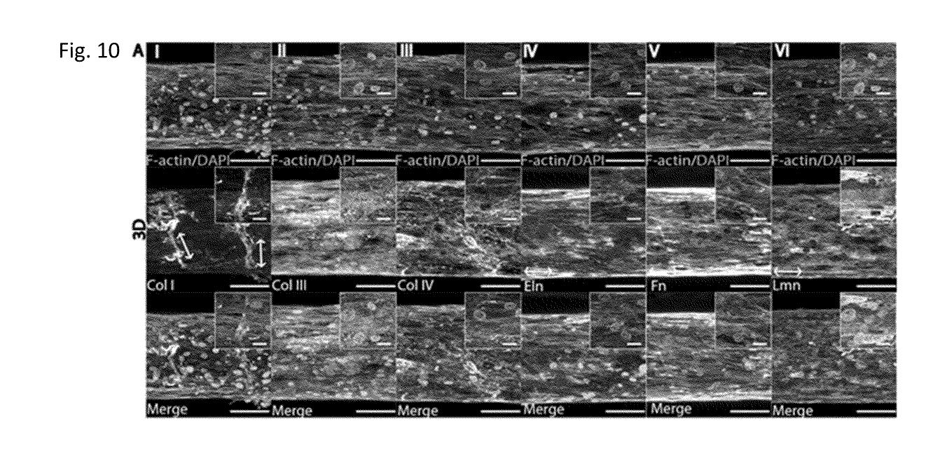

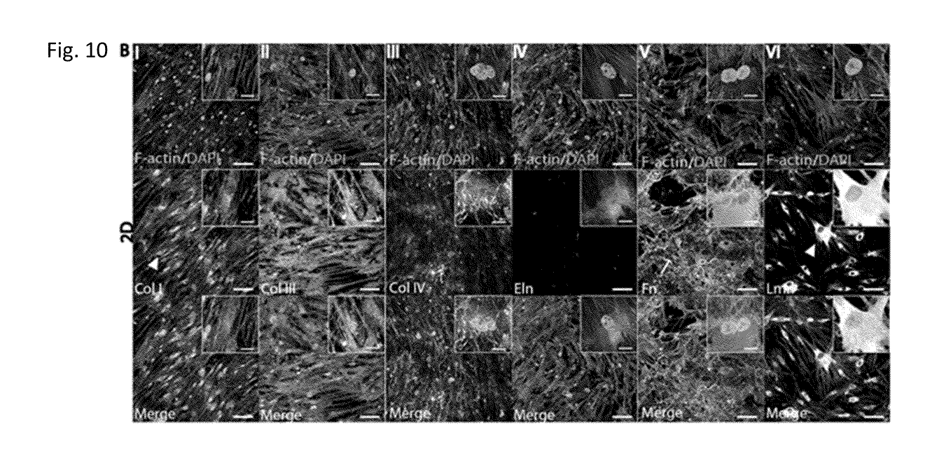

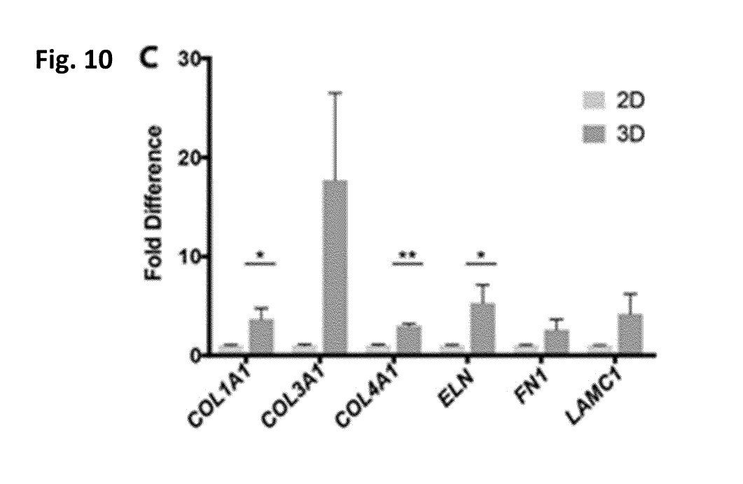

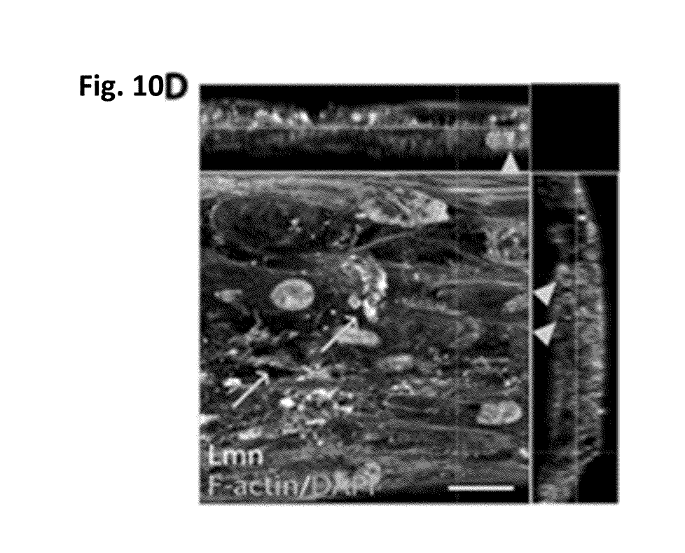

Previously in Example 6 we demonstrated that vSMCs can be introduce on ECFC-seeded fibrin microfibers and obtain full mural cell investment on the developing microvascular structures. Additionally, vSMC ECM proteins Eln and Col 1 were deposited between the ECFC and vSMC layer (Example 6). Here, the three-dimensionality of the fibrin microfibers induces a higher ECM deposition by vSMCs than 2D cultures. As seen in FIG. 10A, SMCs can attach and grow directly on the microfibers.

Furthermore, vSMCs deposit Col I, III, IV, Eln, Fn, and Lmn both in 2D and 3D cultures (FIG. 10A-C). Remarkably, Col I can present a wrapping orientation similar to the previously demonstrated deposition of Col IV by ECFCs when cultured in 3D, compared to a sparse mostly intracellular expression in 2D (FIGS. 10A1 and 10BI). Additionally, Eln, Fn, and Lmn can follow an aligned deposition with cellular orientation along the longitudinal axis of the microfiber in 3D (FIG. 10A IV-VI). In contrast, Fn and Lmn presented a random or intracellular expression in 2D, respectively (FIG. 10B V-VI).

Regarding ECM quantity, Col IV and Eln were upregulated in 3D cultures, with Eln deposition being almost null in 2D but uniformly deposited in 3D (FIGS. 10A IV and 10B IV). Indeed, all proteins were found to be expressed in higher quantities in 3D than in 2D via RT-PCR, though COL1A1, COL4A1, and ELN were the only genes significantly upregulated with average fold differences of 3.7, 3.0, and 4.9 compared to 2D, respectively (FIG. 10C). Similarly to pericytes, vSMCs in 3D grow in a multilayer organization and deposit polymerized Lmn extracellularly (FIG. 10D).

Fibrin Microfiber Degradation

The fibrin microfiber core was degraded after endothelial cell (EC) layer formation while maintaining both cellular viability and intact ECM organization. Fibrin microfibers without cells were treated with different concentrations of plasmin. Plasmin effectively degraded the microfibers in a concentration dependent manner; microfibers treated with a range of concentrations for 24 hrs revealed that 15, 1, 0.25, and 0.1 CU/mL plasmin degraded the microfibers in about 1, 6, 12, and 24 hrs, respectfully (FIG. 11A). Fetal bovine serum present in regular culture media was found to interfere with the degradation process (data not shown), and therefore all plasmin treatments were performed in serum-free media.

Degradation treatment maintained ECFC viability after treating confluent layers of ECFCs with the same conditions found for 1, 6, 12, and 24 hr degradation times. While both 1 hr and 6 hr treatments resulted in poor cell viability (data not shown), the 12 hr and 24 hr treatments resulted in 80.6±4.3% and 65.2±1.8% live cells, respectively (FIG. 11B), compared to 90.7±7.4% when cells were cultured in regular media (not shown).

We then tested the 12 and 24 hr degradation treatments on ECFC-seeded fibrin microfibers that had been in culture for 5 days. As shown in FIG. 11C, the previously demonstrated circumferential organization of ECM proteins Col IV, Fn, and Lmn was maintained after microfiber degradation in both 12 hr and 24 hr treatment conditions (FIGS. 11C I and II respectively). Insert in FIG. 11C II shows a cross-sectional view, demonstrating the resulting lumen.

Luminal Multicellular Microvascular Structure

Multicellular microvascular structures were prepared by culturing ECFCs on fibrin microfibers for 5 days, introducing pericytes and/or vSMCs on top of the endothelial monolayer, and continuing culture for 5 more days. As shown in FIG. 12A-B, the resulting structures contain both an endothelial monolayer expressing the tight junction protein vascular endothelial cadherin (VEcad) and a fully invested perivascular multicellular layer expressing vSMC and pericyte marker SM22. Moreover, abundant ECM protein deposition was observed, including ECM proteins deposited by both ECFCs and mural cells as shown above (Col IV and Fn; FIGS. 12A III and 12B III), mural cells only (Col III; FIG. 12A III), and vSMCs only (Eln, FIG. 12B III). The resulting structures had a cell-ECM wall thickness of 19.9±3.1 μm and 13.6±0.6 μm for pericyte and vSMC co-cultures, respectively.

Constructs were treated with 0.25 CU/mL plasmin for 12 hrs and structures analyzed for lumen formation. As shown in the orthogonal views and 3D reconstructions of confocal microscopy images in FIG. 12C-D, both pericyte and vSMC co-cultures resulted in a distinct circular lumen comprised by ECs and perivascular cells, as evidenced by VEcad and SM22 expression. Furthermore, the structures maintained the ECM protein composition, as shown for Col III and IV (FIG. 12C) and Fn and Eln (FIG. 12D). Orthogonal projections are shown for top Z-stack slices in FIG. 12C and FIG. 12D. The 3D reconstructions show an angle view of half of the resulting cylindrical microvascular structure.

Due to confocal microscopy limitations when imaging large multicellular structures only one halve of the structure is visible (the side closest to the objective), resulting in a semi-circular cross-section. Samples were therefore flipped over and imaged on both sides to confirm structure uniformity, and images presented are representative images for both sides of the structures.

In describing the present invention and its various embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification are incorporated by reference as if each had been individually incorporated.

We claim:

1. A tubular polymer microfiber comprising aligned, electrostretched polymer nanofibers, wherein the microfiber has a longitudinally aligned nanotopography.

2. The microfiber of claim 1, wherein the polymer microfiber has a diameter from about 100 μm to about 500 μm.

3. The microfiber of claim 1, wherein the polymer is selected from alginate, fibrin (fibrinogen), gelatin, hyaluronic acid, or combinations thereof.

4. The microfiber of claim 3, wherein the polymer is fibrin.

5. The microfiber of claim 1, further comprising endothelial progenitor cells seeded on the polymer microfiber.

6. The microfiber of claim 5, wherein the endothelial progenitor cells are endothelial colony forming cells.

7. The microfiber of claim 6, wherein the endothelial colony forming cells are aligned longitudinally to the polymer microfiber.

8. The microfiber of claim 7, wherein the endothelial colony forming cells deposit extracellular matrix proteins, and wherein the extracellular matrix proteins are circumferentially organized, wrapping around the microfiber.

9. The microfiber of claim 8, wherein the extracellular matrix proteins include laminin, collagen IV, and fibronectin.

10. The microfiber of claim 9, wherein collagen IV, laminin, and fibronectin are deposited in higher quantities on the microfiber than on 2D cultures.

11. The microfiber of claim 1, further comprising perivascular cells seeded on the polymer microfiber.

12. The microfiber of claim 11, wherein the perivascular cells are pericytes.

13. The microfiber of claim 12, wherein the pericytes deposit extracellular matrix proteins, and wherein the extracellular matrix proteins are longitudinally organized along the microfiber.

14. The microfiber of claim 13, wherein the extracellular matrix proteins include collagen types I, III, IV, laminin, and fibronectin.

15. The microfiber of claim 14, wherein collagen types I, III, IV, laminin, and fibronectin are deposited in higher quantities on the microfiber than on 2D cultures.

16. The microfiber of claim 11, wherein the perivascular cells are vascular smooth muscle cells.

17. The microfiber of claim 16, wherein the vascular smooth muscle cells deposit extracellular matrix proteins, and wherein the extracellular matrix proteins are longitudinally, randomly, or circumferentially organized along the microfiber.

18. The microfiber of claim 17, wherein the extracellular matrix proteins include collagen types I, III, IV, elastin, laminin, and fibronectin.

19. The microfiber of claim 18, wherein collagen types I, III, IV, elastin, laminin, and fibronectin are deposited in higher quantities on the microfiber than on 2D cultures.

20. The microfiber of claim 5, further comprising a second cell type seeded on the fibrin microfiber.

21. The microfiber of claim 20, wherein the second cell type is a mural cell.

22. The microfiber of claim 21, wherein the mural cell is vascular smooth muscle cell or a pericyte.

23. The microfiber of claim 22, wherein the vascular smooth muscle cell or the pericyte encircles, is randomly oriented, or is longitudinally oriented with respect to the fibrin microfiber.

24. The microfiber of claim 23, wherein the vascular smooth muscle cell deposits collagen type I and elastin, and the pericyte deposits collagen type IV.

25. A microvascular structure comprising the polymer microfiber of claim 5.

26. The microvascular structure of claim 25, further comprising a mural cell.

27. The microvascular structure of claim 26, wherein the mural cell is a vascular smooth muscle cell or a pericyte.

28. The microvascular structure of claim 27, wherein the vascular smooth muscle cell deposits collagen type I and elastin, and the pericyte deposits collagens type III and IV.

29. The microfiber of claim 8, wherein the fibrin microfiber is degraded.

30. The microfiber of claim 29, wherein the degradation is performed with plasmin.

31. A method of degrading the polymer microfiber of claim 8 with varying concentrations of plasmin.

32. The method of claim 31, wherein the cells maintain viability.

33. The method of claim 31, wherein the extracellular protein organization is maintained after degradation.

34. The microvascular structure of claim 28, wherein the polymer microfiber is degraded.

35. A method of sequentially controlling microvascular vessel formation comprising the steps of:
a. preparing the polymer microfiber of claim 1;
b. seeding the microfiber with endothelial progenitor cells; and
c. co-culturing the endothelial cell-seeded microfiber with a perivascular cell, wherein the cells deposit extracellular matrix proteins that encircle the microfiber, and are oriented perpendicular to the cell orientation, along the fiber's circumference, and wherein formation of microvasculature vessel is sequentially controlled.

36. The method of claim 35, wherein the endothelial progenitor cells are endothelial colony forming cells and the perivascular cells are vascular smooth muscle cells or pericytes.

37. The method of claim 36, further comprising degrading the fibrin microfiber with an enzyme.

38. The method of claim 37, wherein the enzyme is plasmin.

39. A system for sequentially controlling microvascular vessel formation comprising:
a. the electrostretched polymer microfiber of claim 1 for forming a matrix for the culture of cells that form the vasculature;
b. endothelial progenitor cells seeded on the microfiber for forming a vascular endothelium;
c. vascular smooth muscle cells or pericytes co-cultured with the endothelial cell-seeded microfiber for forming a tunica media layer;
d. polymer microfiber degradation post multicellular multilayer vascular structure formation,
wherein the cells deposit extracellular matrix proteins that encircle the microfiber, and are oriented perpendicular to the cell orientation along the fiber's circumference, and wherein a luminal microvascular vessel is formed.
